(19) United States
(12) Patent Application Publication
(10) Pub. No.: US 2022/0220504 A1
(43) Pub. Date: Jul. 14, 2022

(54) Method for Generation of Genetically Modified T Cells

(71) Applicant: Miltenyi Biotec B.V. & Co. KG, Bergisch Gladbach (DE)

(72) Inventors: Thomas Schaser, Bergisch Gladbach (DE); Andrew Kaiser, Bergisch Gladbach (DE); Mario Assenmacher, Bergisch Gladbach (DE)

(21) Appl. No.: 17/612,410

(22) PCT Filed: May 27, 2020
(86) PCT No.: PCT/EP2020/064755

§ 371 Date: Nov. 18, 2021

(30) Foreign Application Priority Data
May 28, 2019 (EP) 19176957.9

(51) Int. Cl. C12N 15/86; C12N 5/0783; C12N 15/62; C07K 14/725; C07K 14/705

(52) U.S. Cl. CPC C12N 15/86; C12N 5/0636; C12N 15/625; C07K 14/7051; C07K 14/70521; C12N 2740/15043; C07K 2319/33; C12N 2510/00; C12N 2501/2307; C12N 2501/2315; C12N 2501/505; C07K 2319/30; C12N 2740/15063

(57) ABSTRACT

The present invention provides a method for the generation of genetically modified T cells comprising the steps a) a sample provided comprising T cells, b) preparation of said sample by centrifugation, c) enrichment of the T cells, d) activation of the T cells using modulatory agents, e) genetic modification of the T cells by transduction with lentiviral vector particles, f) removal of said modulatory agents, thereby generating a sample of genetically modified T cells, wherein said method is performed in equal or less than 144 hours, less than 120 hours, less than 96 hours, less than 72 hours, less than 48 hours, or less than 24 hours. In one embodiment of the invention said enrichment of T cells is performed by magnetic cell separation using magnetic particles that are directly or indirectly coupled to antibodies or antigen binding fragments thereof specific for CD4 and/or CD8 wherein said magnetic particles can be removed from the cells after separation.

### FIELD OF INVENTION

The present invention relates to the field of the generation of genetically engineered T cells, in particular to the generation of genetically engineered T cells within a short period of time and with low concentration of contaminating substances and/or undesired cells in the target population.

### BACKGROUND OF THE INVENTION

The clinical manufacture of gene-modified T cells is a complex process. Patient's peripheral blood mononuclear cells (PBMCs) are often enriched for T cells and activated prior to gene modification with viral or nonviral vectors. The modified T cells are then expanded in order to reach the cell numbers required for treatment, after which the cells are finally formulated and/or cryopreserved prior to reinfusion. The cell product must be subjected to a number of quality control assays and has to meet all release criteria and Good Manufacturing Practices (GMP) guidelines. Thus far, adoptive cell transfer (ACT) using gene-modified T cells has often been carried out by investigators who have developed their manufacturing process for small scale clinical trials by using the devices and infrastructure at hand. Meanwhile automated processes in closed systems are also available (e.g. WO2015162211A1, WO2019046766A1). In WO2019032929A1 a method for genetically engineering T cells is disclosed, wherein a sample comprising T cells is incubated under stimulating conditions and wherein a nucleic acid is introduced into the stimulated T cells at least during a portion of said incubating.

There is a need in the art for improved methods for generation of genetically modified T cells, preferentially automated processes, for example to reduce toxicity and/or reduced processing time of the generated T cells, to allow, for example, improved administration to patients in need thereof.

### SUMMARY OF THE INVENTION

Remaining modulatory agents contaminating the drug product may be harmful upon infusion as they may lead to unwanted activation of T cells in vivo. This may lead to a rapid release of proinflammatory cytokines, causing severe cytokine release syndrome, fever, hypotension, organ failure and even deaths. In addition, remaining lentiviral vectors contaminating the drug product in soluble and/or cell bound form may be harmful upon infusion as they may provoke an unwanted immune response such as complement activation, antibody-dependent cell-mediated cytotoxicity, inducing an adoptive immune response against antigens delivered by the lentiviral vector and/or transduction of non-target cells in vivo. The transduction of non-target cells and the subsequent expression of the transgene may induce unwanted side-effects such as the induction of unwanted immune responses, oncogenicity, altered survival, proliferation, physiological state and natural function.

Surprisingly, it was found that the process of generating modified T cells as disclosed herein can be reduced to less than 144 hours, less than 120 hours, less than 96 hours, less than 72 hours, less than 48 hours, or even less than 24 hours from the beginning of the process, when molecules, reagents potentially hazardous to the patient are removed during and/or at the end of the process as cleanup and additional layer of safety i.e. the provision of a sample that comprises T cells, to the sample that comprises the genetically modified T cells that subsequent may be ready to (re)-infusion to a patient in need thereof. The genetically modified T cells may be T cells that express a chimeric antigen receptor and the application may be for treating cancer in a patient.

It was surprising that there is no need to expand in-vitro the engineered T cells to cell numbers that have been known to be required for effective treatment in a patient as the further expansion of these genetically T cells to therapeutic effective amounts of cells will take place in vivo. The expansion of the number (amount) of genetically modified T cells in the generated sample as disclosed herein may be less than 10-fold, preferentially less than 5-fold compared to the number (amount) of T cells of the provided sample at the begin of the process. This is possible due to the high quality of composition/sample of genetically modified T cells generated by the method as disclosed herein, i.e. the low contamination with reagents, lentiviral vectors and non-engineered T cell components.

The present invention successfully demonstrates that CAR T cells in-vitro generated within few days, e.g. in equal or less than 3 days (72 hours) using the method as disclosed herein in the absence of an explicit expansion step surprisingly promote robust antitumoral activity in vitro and in vivo proving that in vivo expansion but not in vitro expansion is essential for the generation of functional CAR T cells (see Example 10).

The data have been shown for the method performed in 3 days but it is self-explaining that the in-vivo effect will be observed also with a generated sample of said method in less than 72 hours (3 days), e.g. 48 hours or 24 hours, merely the duration of triggering the in-vivo effect of killing the cancerous cells by the generated cells will be delayed. FIG. 9 provides data indicating the manufacturing time may be reduced even further with an only reduction in gene transfer efficiency.

### BRIEF DESCRIPTION OF THE DRAWINGS

FIG. 1: Schematic representation for the generation of genetically modified T cells in a short period of time

A sample is provided containing T cells such as whole blood of a human, leukapheresis, buffy coat, PBMC, outgrown or isolated T cells. Optionally, the sample contains serum containing substances inhibiting the genetic modification by lentiviral vectors. To enable efficient transduction serum is removed by washing. In addition, T cells are polyclonally activated with a modulatory agent binding to CD3 and CD28 and subsequently genetically modified using lentiviral vectors. As cleanup, the modulatory agent is removed to obtain purified genetically engineered T cells.

FIG. 2: Schematic representation for the removal of the modulatory activating agent

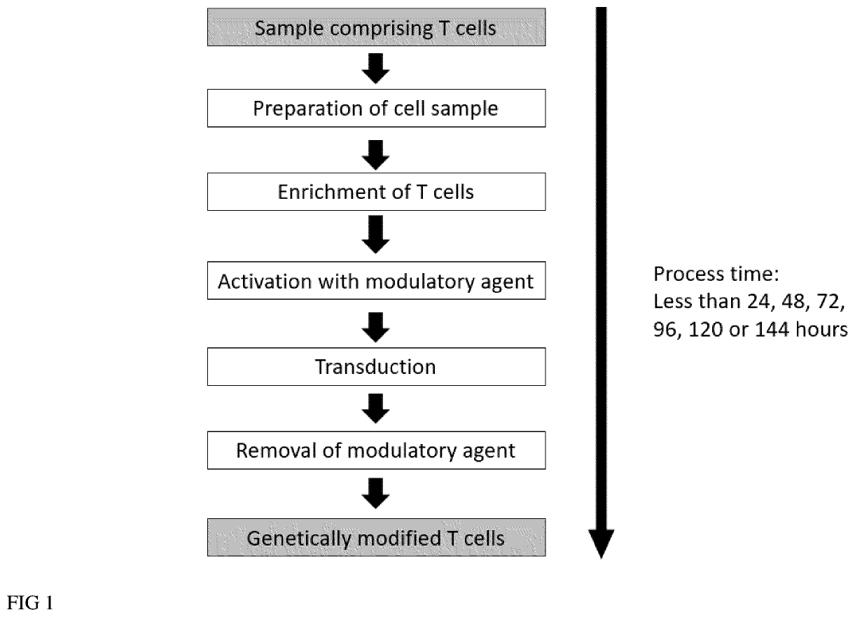

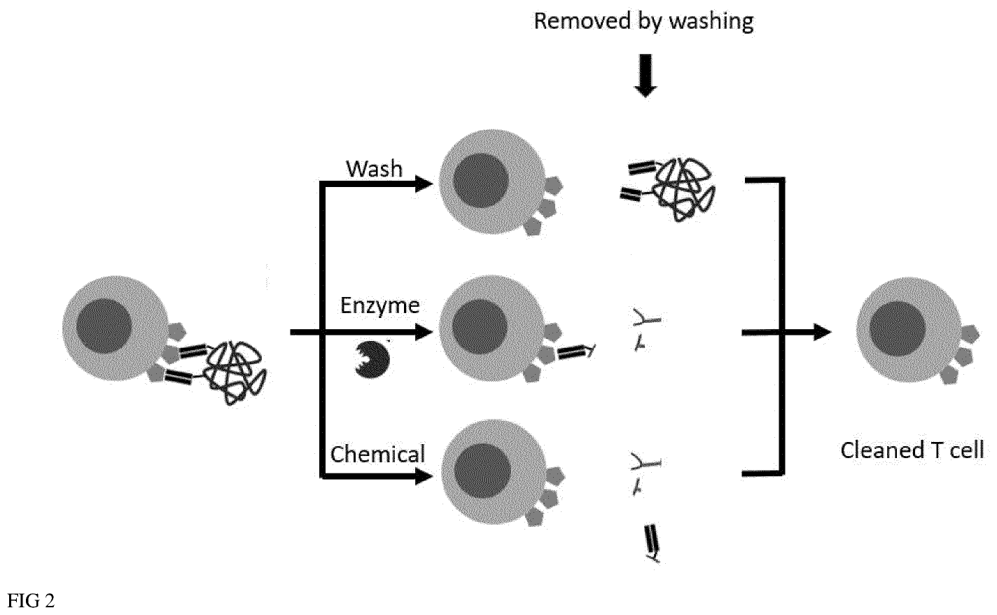

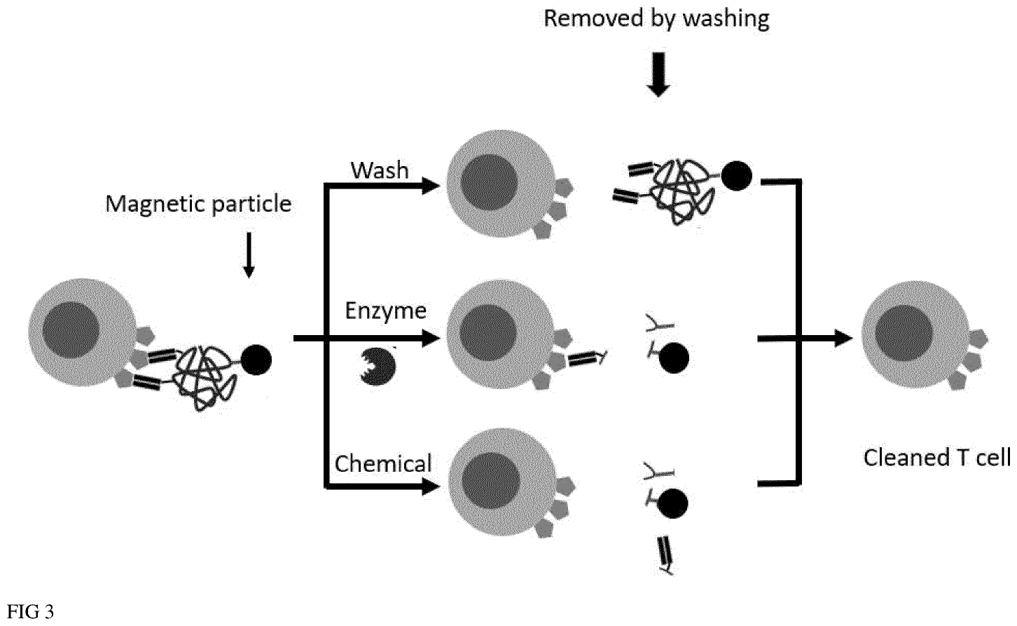

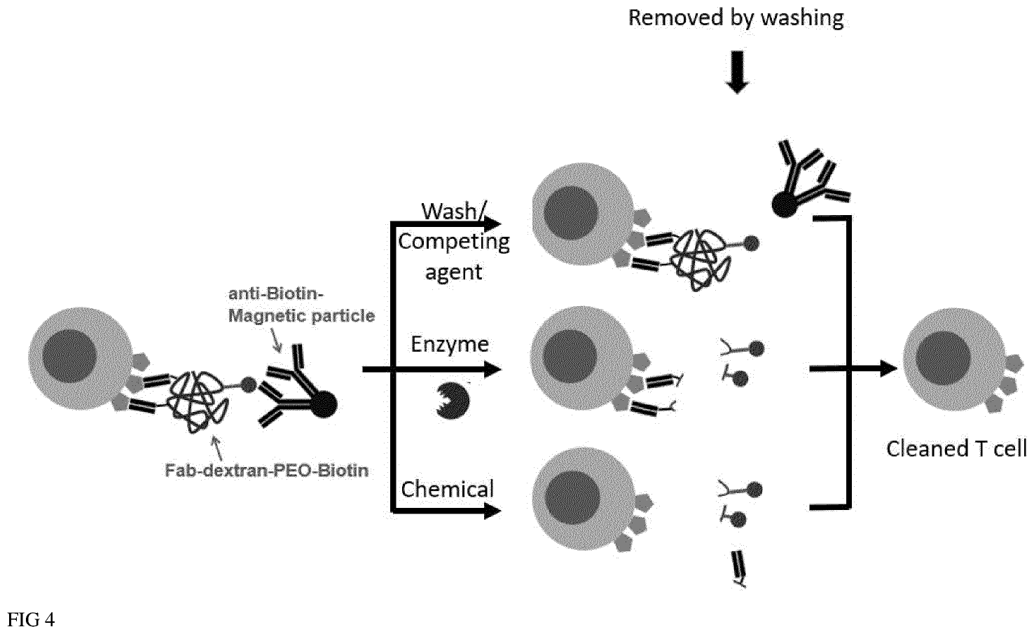

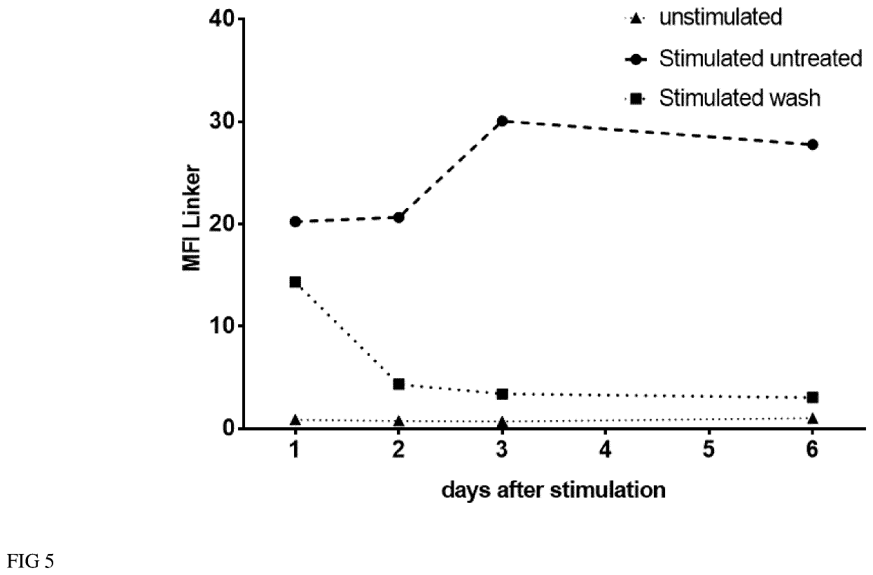

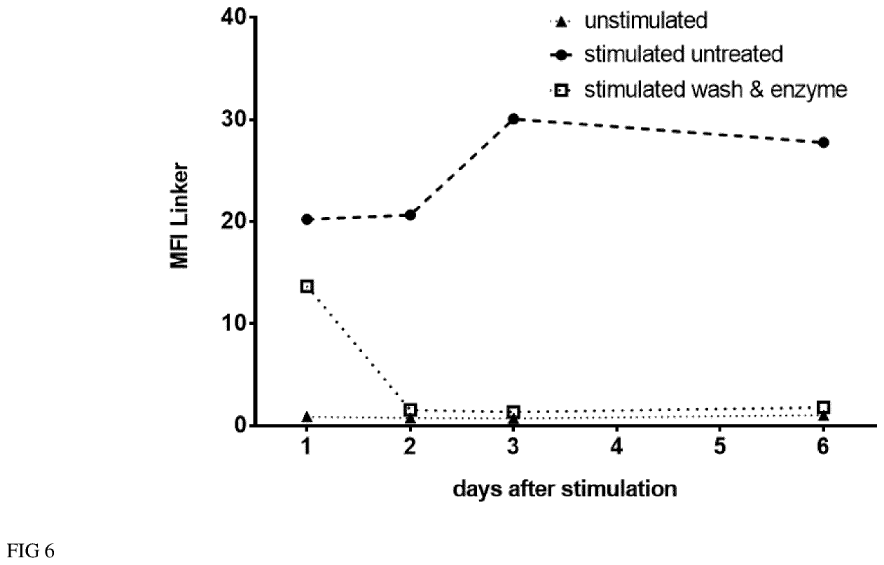

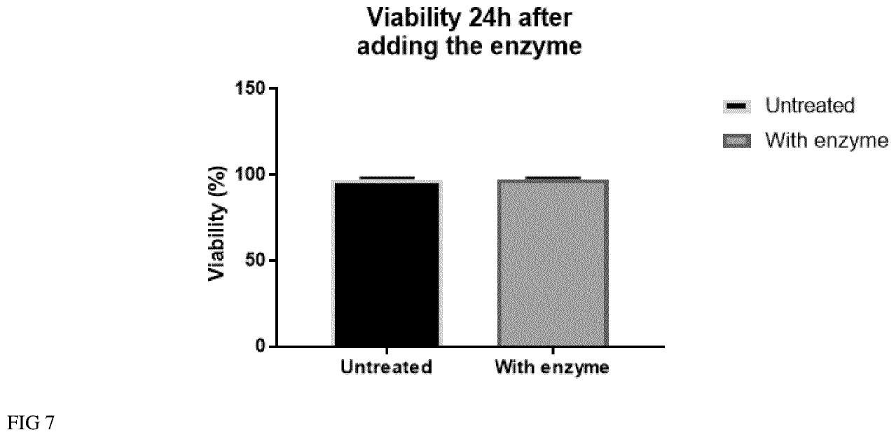

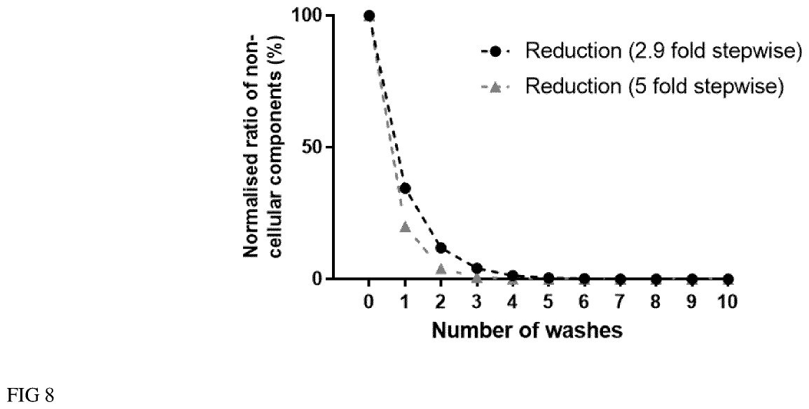

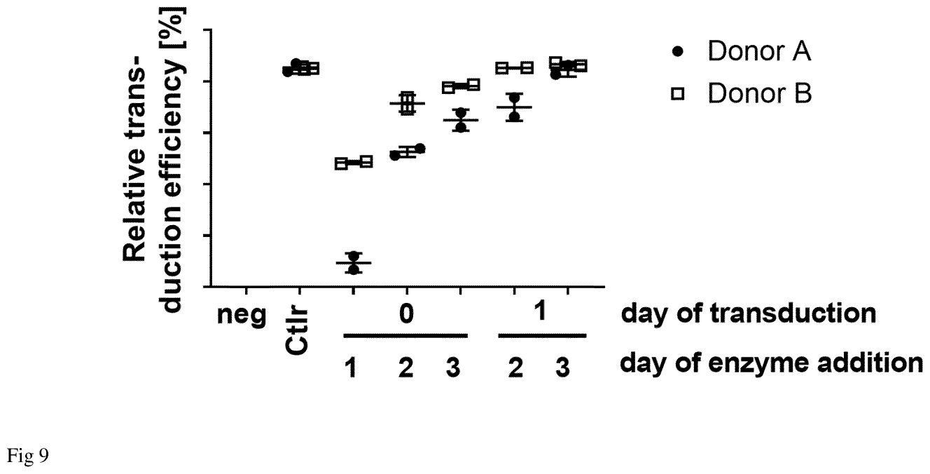

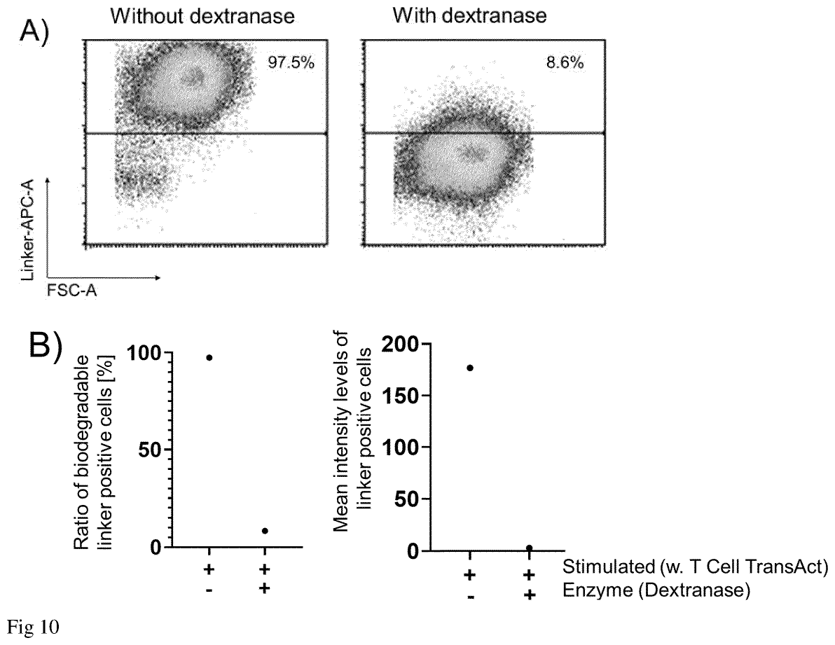

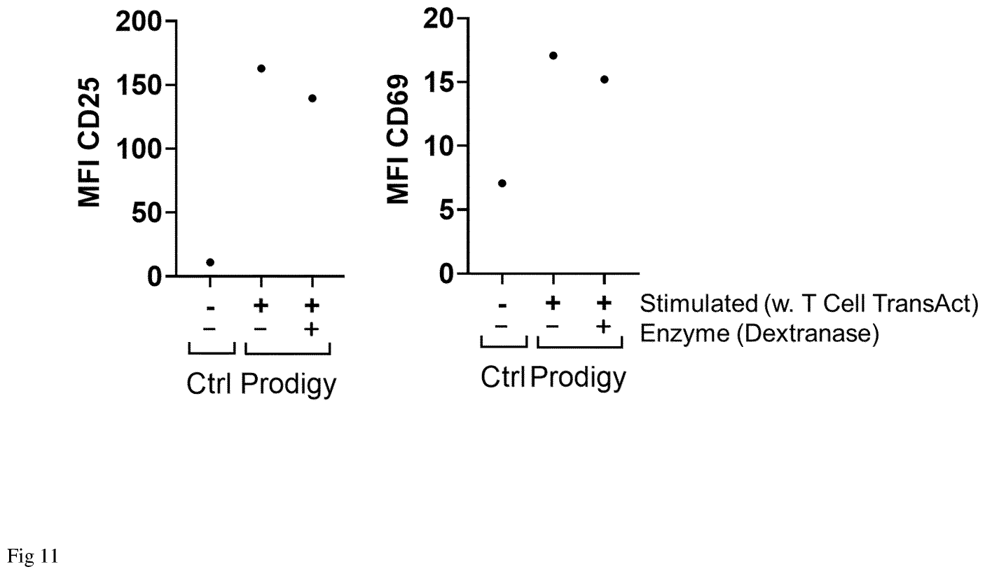

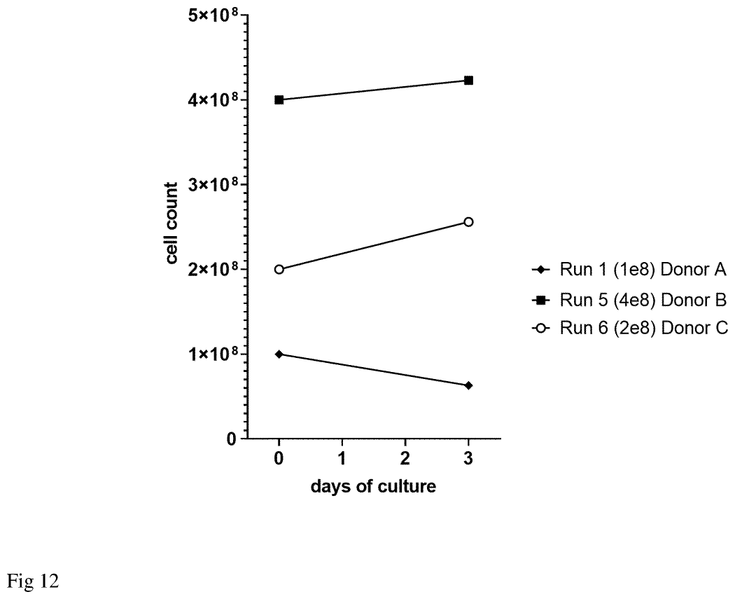

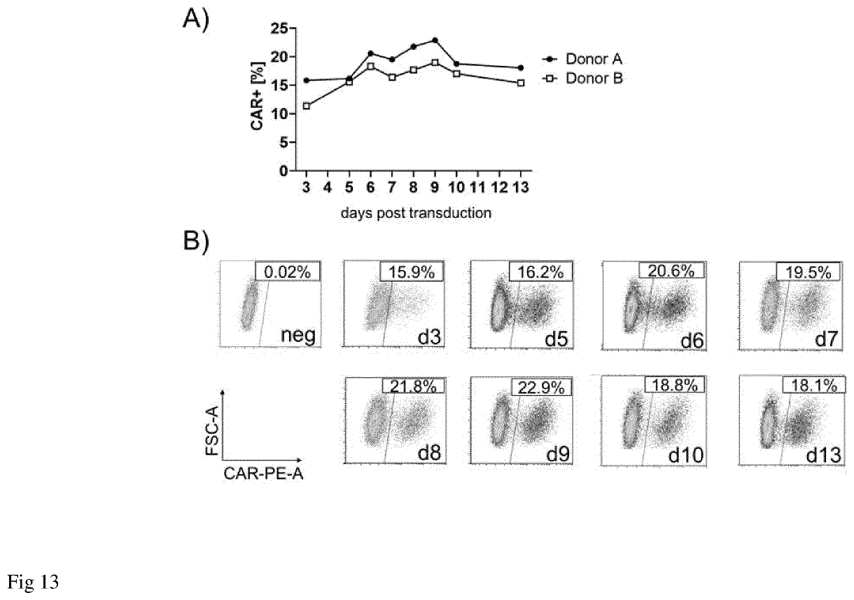

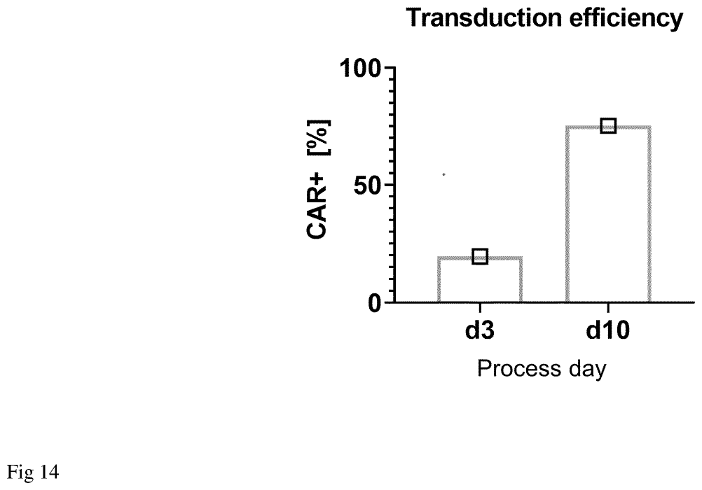

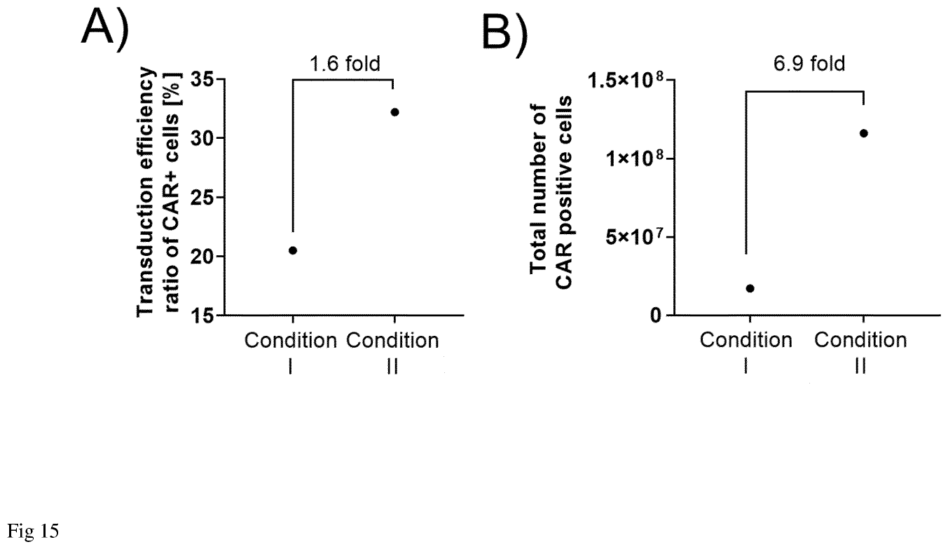

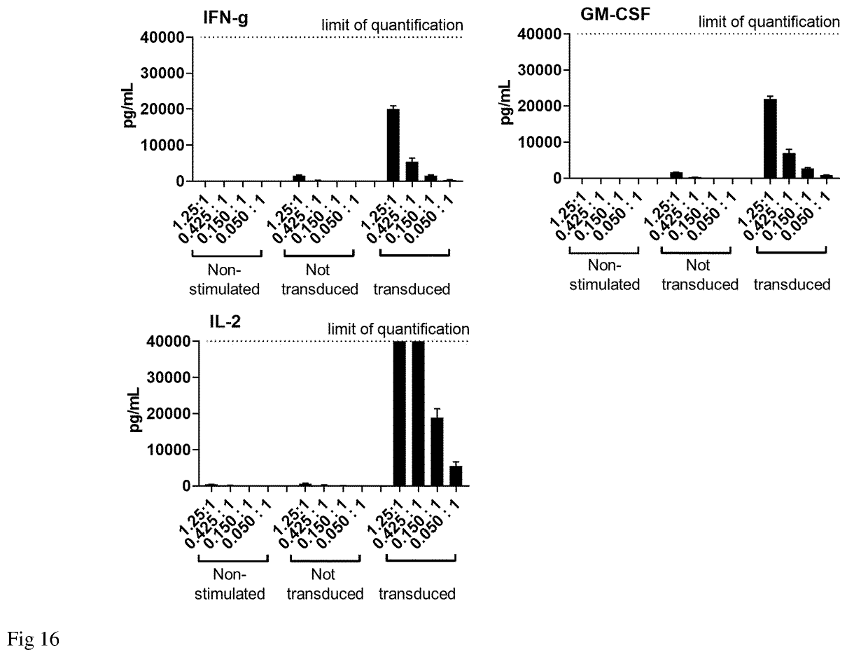

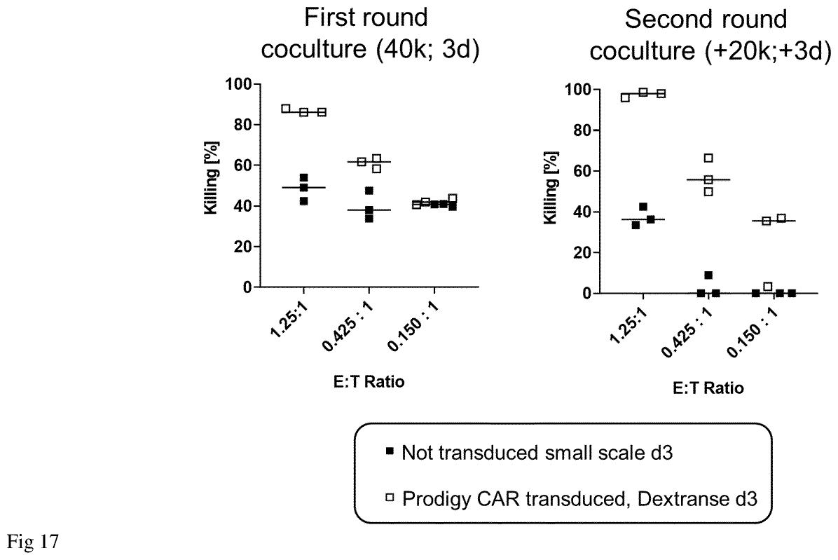

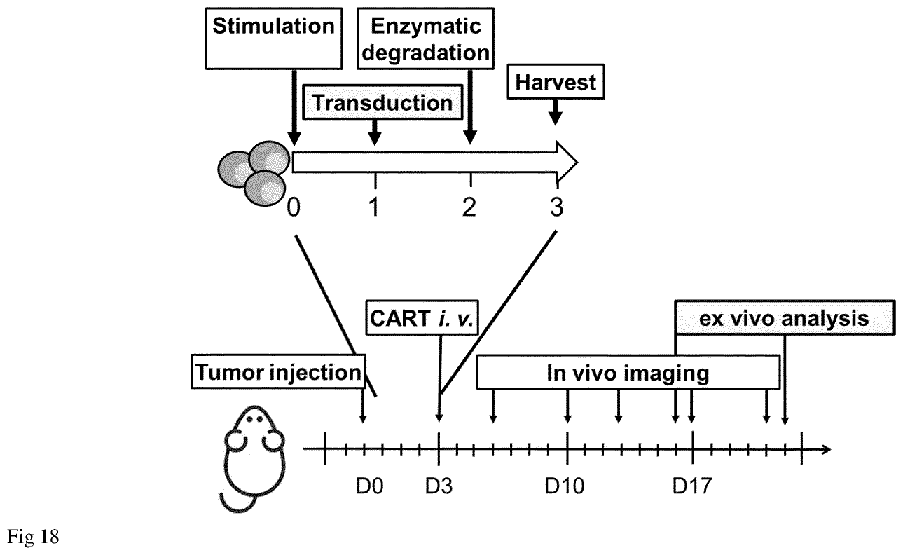

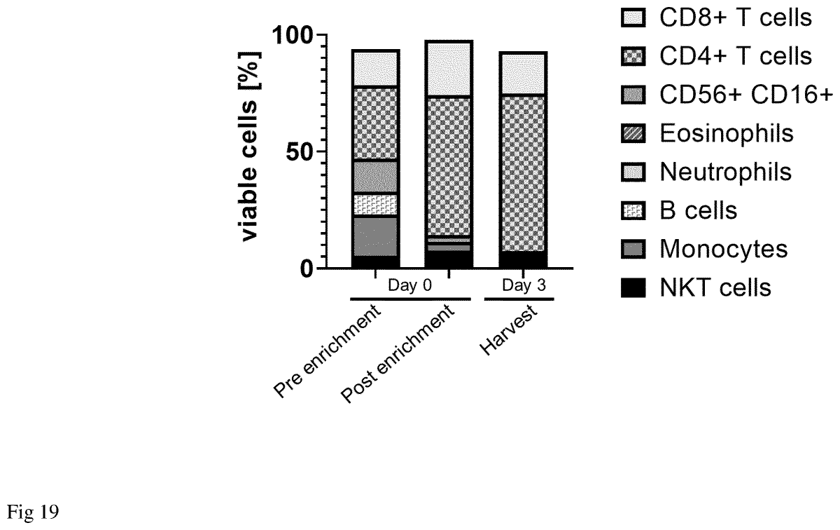

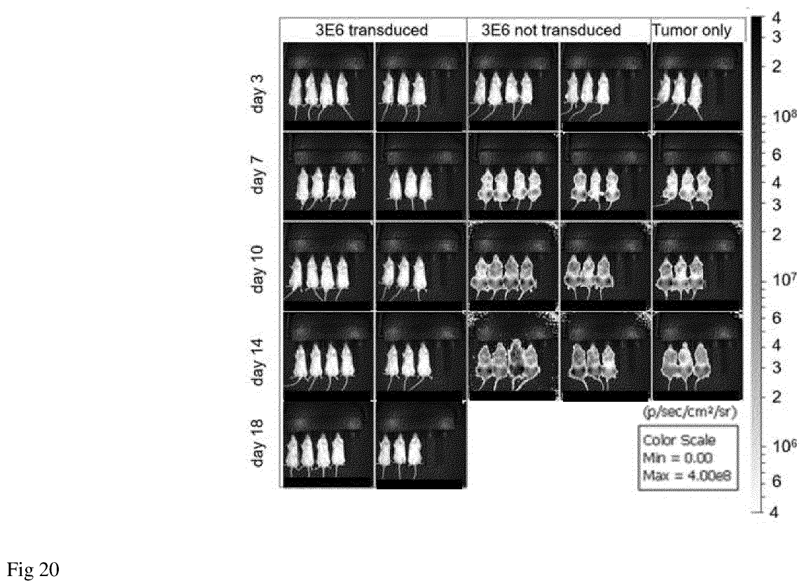

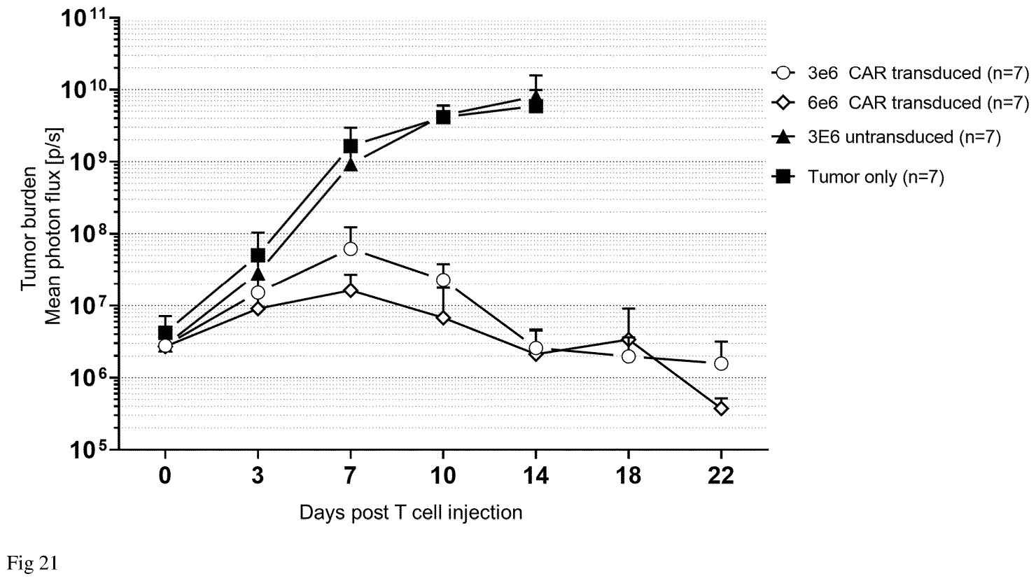

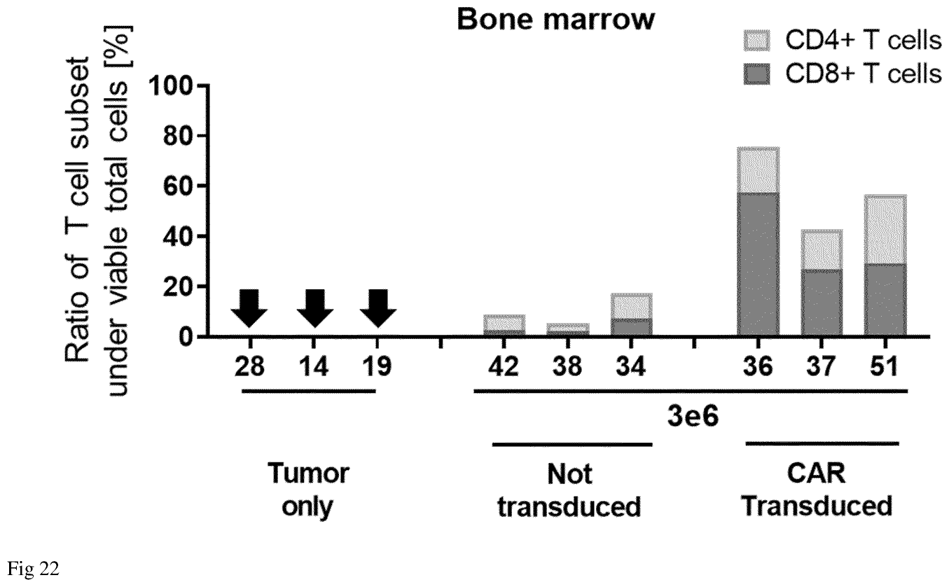

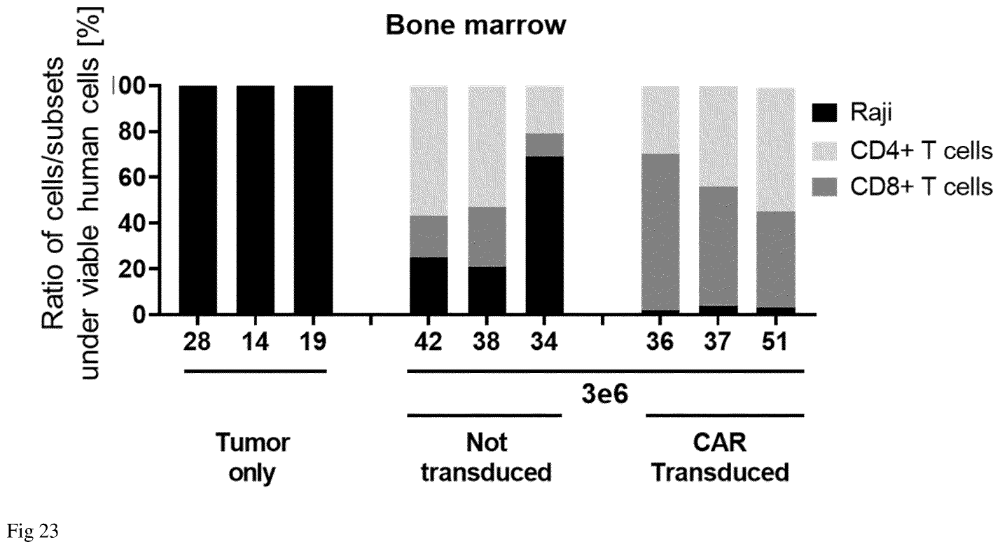

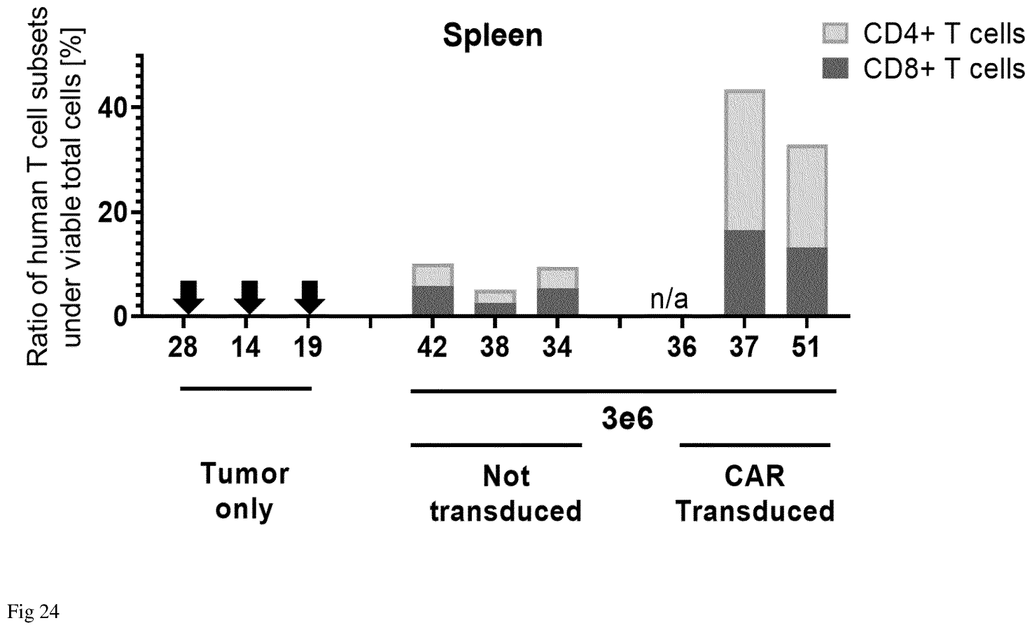

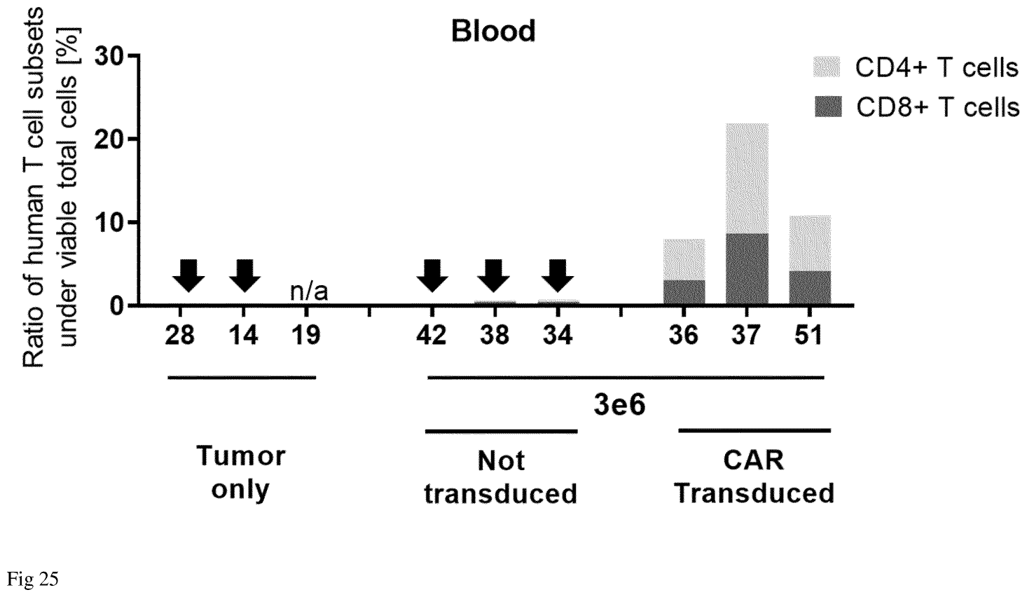

T cells are polyclonally activated with a modulatory reagent comprising an antibody or antigen binding fragment thereof specific for CD3 and an antibody or antigen binding fragment thereof specific for CD28. Both antibodies or fragments thereof are coupled directly or indirectly to a biodegradable linker. The modulatory activating reagent may be removed by washing or by adding enzymes specifically degrading the linker, thereby the antibodies or fragments specific for CD3 and CD28 are released. In addition, the activating reagent may be removed by chemical disruption of said antibodies or antigen binding fragments thereof specific for CD3 and CD28. Removal of the modulatory agent or fragments thereof from the cells may be performed by one or several washing steps.

FIG. 3: Schematic representation for the removal of the magnetic enrichment reagents

CD4+ and/or CD8+ T cells are separated by magnetic cell separation such as MACS with magnetic particles directly or indirectly contacting T cells with coupled antibodies or antigen binding fragments thereof specific for CD4 and/or CD8. The antibodies or antigen binding fragments thereof are coupled to the magnetic particles via a biodegradable linker. The coupled magnetic particles may be removed by washing or by adding enzymes specifically degrading the linker, thereby the antibodies or fragments specific for CD4 and/or CD8 are released from the magnetic particle. In addition, the magnetic particle may be removed by chemical disruption. Removal of the magnetic particle or fragments thereof may be performed by one or several washing steps.

FIG. 4: Schematic representation for the removal of reagents for the indirect magnetic labelling of T cells.

T cells may be indirectly labelled with a magnetic particle contacting T cells with coupled antibodies or antigen binding fragments thereof specific for CD4 and/or CD8 via a biodegradable linker that is biotinylated and a magnetic particle that is coupled to an antibody or antigen binding fragment thereof specific for biotin. The magnetic particle may be released from said T cell by adding an enzyme that specifically digests the biodegradable linker and/or by adding biotin (as a competitor). In addition, the indirectly coupled magnetic particle may be removed by washing and/or chemical disruption. Removal of disrupted agents or the magnetic particle may be performed by one or several washing steps.

FIG. 5: Removal of the activating reagent by washing Enriched T cells were polyclonally stimulated with T Cell TransAct™ (Miltenyi Biotec)—a modulatory reagent comprising an antibody or antigen binding fragment thereof specific for CD3 and an antibody or antigen binding fragment thereof specific for CD28 coupled directly to a biodegradable linker. 20h post stimulation T cells containing the modulatory activating reagent were washed and the presence of bound biodegradable linker was measured by flow cytometry at several timepoints post stimulation. Washing removes the stimulation reagent efficiently as detected by reduced levels of the biodegradable linker over time.

FIG. 6: Removal of the modulatory agent by washing and enzymatic activity

Enriched T cells were polyclonally stimulated with T Cell TransAct™ (Miltenyi Biotec)—a modulatory reagent comprising an antibody or antigen binding fragment thereof specific for CD3 and an antibody or antigen binding fragment thereof specific for CD28 coupled directly or indirectly to a biodegradable linker. 20h post stimulation T cells with bound modulatory activating reagent were washed and the presence of the biodegradable linker was measured by flow cytometry after 24h. 26h post stimulation the enzyme specific for the biodegradable linker was added and presence of the biodegradable linker was measured over time at several timepoints post stimulation. Washing and the addition of the enzyme specific for the biodegradable linker removes the stimulation reagent efficiently.

FIG. 7: The enzyme specific for the biodegradable linker is non-toxic

Enriched T cells were polyclonally stimulated with T Cell TransAct™ (Miltenyi Biotec)—a modulatory reagent comprising an antibody or antigen binding fragment thereof specific for CD3 and an antibody or antigen binding fragment thereof specific for CD28 coupled directly or indirectly to a biodegradable linker. 26h post stimulation the enzyme was added to the T cells and 24h later the viability was measured by PI staining by flow cytometry. The enzyme specific for the biodegradable linker does not harm the enriched and activated T cells as comparable viabilities were detectable with and without the enzyme specific for the biodegradable linker.

FIG. 8: Efficient removal of non-cellular components by cumulative washing

The efficiency of cumulative washing and removal of non-cellular components was calculated based on two different washing regimen: either 2.6 fold dilution per individual washing step or 5 fold dilution per individual washing step. The calculated cumulative dilution efficiency was normalized to undiluted (i.e. 100%). For 2.6-fold step wise dilution the ratio of non-cellular components falls below 0.001% after 11 consecutive washing steps. For 5-fold step wise dilution the ratio of non-cellular components falls below 0.001% after 7 consecutive washing steps.

FIG. 9: Setting up the process for the genetic engineering of T cells in 3 days

T cells transduced on day 0 and incubated with dextranase on day 1—a enzyme specific for the biodegradable linker—showed the lowest transduction efficiency levels indicating insufficient T cell stimulation. This was confirmed by analyzing T cells that were stimulated longer by adding later on day 2 or 3 and higher transduction efficiency levels were detectable as compared to T cells incubated with the enzyme on day 0. Better transduction efficiencies that were close to the conventional protocols were observed for stimulated T cells that were transduced on day 1 and incubated with dextranase on day 2 or 3.

FIG. 10: Efficient removal of the modulatory agent in CliniMACS® Prodigy system for genetically engineered T cells generated within 3 days

A leukapheresis sample of a healthy donor with up to 1e9 CD4/CD8 cells was automatically processed in the CliniMACS® Prodigy system to generate CAR T cells within 3 days. 4e8 T cells were polyclonally stimulated with the modulatory agent MACS® GMP T Cell TransAct™ (Miltenyi Biotec) and genetically modified with VSV-G pseudotyped lentiviral vectors. On day 2, 10 ml of a solution containing dextranase was automatically added specifically degrading the biodegradable linker releasing the antibodies or fragments specific for CD3 and CD28 and abolishing the activity of the modulatory agent. As control, a manufacturing run in the CliniMACS® Prodigy system was performed under the same conditions and the same donor material but without the addition of the enzyme specific for the biodegradable linker.

FIG. 10A: The presence of the biodegradable linker was assessed for both T cell engineering runs in the CliniMACS® Prodigy system by flow cytometry on the formulated cells by staining with antibodies specific for the biodegradable linker.

FIG. 10B: The biodegradable linker was efficiently removed in the CliniMACS® Prodigy system as only a minor fraction of linker positive cells was detectable when compared to the CliniMACS® Prodigy run without added enzyme. In addition, the mean intensity levels (MFI) for the biodegradable linker for all viable cells was at background levels when the enzyme was added.

FIG. 11: T cell stimulation levels in the CliniMACS® Prodigy system upon enzymatic removal of the activation reagent on day 2

The impact of removing the modulatory agent on the stimulation levels was evaluated by flow cytometry upon staining for CD25 and CD69 as both are described to be reliable T cell activation markers (CD25: REA570; CD69: REA824; Miltenyi Biotec). Non-stimulated T cells obtained from the same donor from small scale cultures served as control and harvested T cells from the CliniMACS® Prodigy system treated with or without enzyme were analyzed.

Compared to the non-stimulated control cells, highly elevated mean intensity levels for both activation markers were detected for T cell samples treated with or without dextranase confirming that the stimulation until day 2 was already sufficient to upregulation of both activation markers. This also indicates that the modulatory agent may be removed already at day 2 or even earlier without affecting the stimulation.

FIG. 12: Proliferation of stimulated T cells in the CliniMACS® Prodigy system for the genetic engineering of T cells within 3 days.

T cell expansion was not detectable on day 3 for three independent manufacturing runs suggesting that the T cells were sufficiently activated but proliferation of T cells has not started yet (see also FIG. 12). In consequence, the manufacturing protocol for the genetic modification of T cells within 3 days is too short to support T cell proliferation in vitro.

FIG. 13: Evaluating the CAR expression kinetics in small scale

FIG. 13A: After day 5 the transduction efficiency reached plateau levels at 18-22% confirming stable transgene delivery and transgene expression.

FIG. 13B: 2 days post transduction 16% of the T cells were already CAR positive but a distinct CAR positive population was not detectable yet. At later time points a distinct CAR expressing population was detected by flow cytometry.

FIG. 14: Evaluating the CAR expression kinetics for the large scale manufacture in the CliniMACS® Prodigy system

In contrast to the experiments in small scale (see FIG. 13), the plateau level of CAR expression in the CliniMACS® Prodigy system were not reached at early time points. 2 days post transduction 19% of the T cells were CAR positive. Transduction efficiency increased to 75% at later time points indicating that the CAR was not yet sufficiently expressed 2 days post transduction.

FIG. 15: Optimizing CAR T cell manufacturing parameters in the CliniMACS® Prodigy system Isolated and stimulated T cells were genetically modified with 2.5 ml of VSV-G pseudotyped CD20/CD19 tandem CAR encoding lentiviral vectors for 1e8 T cells (see FIG. 15: Condition I) and in parallel with the same lentiviral vector volume for 4e8 T cells (see FIG. 15: Condition II). Condition II also supports cultivation at higher cell densities by increasing the volume and by implementing early shaking steps. On day 2, the same volume of dextranase was applied to both T cell manufacturing conditions. On day 3, the manufactured T cells were washed multiple times, harvested and the total T cell number was determined by cell counting. A washed and harvested cellular sample of both CAR T cell manufacturing conditions was cultivated for another 8 days in 24 wells in the incubator to enable a reliable assessment of the transduction efficiency when steady state levels of the CAR expression are typically observed.

FIG. 15A: The transduction efficiency was 32% for condition II, whereas the transduction efficiency for condition I was only 20% Importantly, a higher LV dose per cell (MOI) was applied for condition I.

FIG. 15B: For condition II not only a higher transduction efficiency was determined but also 4 times more T cells (i.e. 4e8) were transduced. This increased the yield of CAR transduced T cells almost 7 fold for condition II when compared to condition I.

FIG. 16: Cytokine expression levels of CAR T cells generated within 3 days

Stimulated, CD20-CAR transduced and with dextranase treated T cells manufactured within 3 days in the CliniMACS® Prodigy system were cocultivated at different effector to target ratios (E:T) with CD20, GFP expressing Raji cells and the presence of inflammatory cytokines such as Interferon-gamma (IFN-g), Granulocyte-macrophage colony-stimulating factor (GM-CSF) and IL-2 was evaluated 24 h later using the MACSPlex Cytokine Kit Assay (Miltenyi Biotec). For CD20 CAR transduced T cells generated within 3 days, IFN-g, GM-CSF and IL-2 levels were detectable at high levels even beyond the level of quantification in an E:T dependent manner. In contrast, no cytokines were detectable for non-stimulated T cells and for stimulated T cells that remained untransduced. This confirms the tumor antigen specific response of CAR transduced T cells that were manufactured within 3 days.

FIG. 17: Cytotoxic activity of CAR T cells generated within 3 days

CAR T cells manufactured within 3 days and Raji-GFP cells were cocultered for another 2 days when 50% of the cells were analyzed by flow cytometry to quantify the number of remaining tumor cells and consequently the cytolytic potential of the CAR T cells (round 1; left). Another 20,000 Raji-GFP tumor cells were added to the remaining 50% of the coculture to evaluate the potency of the CAR T cells in a second consecutive round of coculture when additional tumor cells were added and the cytotoxic activity was assessed under conditions meant to be challenging for the CAR T cells (round 2: right). After 72h flow cytometry was performed to quantify the number of remaining tumor cells of the second round of coculture. For high E:T ratios (i.e. 1.25:1) almost 100% of the Raji cells were lysed in the first and also in the second round. In contrast only 50% and 40% remaining target cells were detectable for the untransduced control. For a E:T ratio of 0.425:1 the functionality was comparable as for 1.25:1 but at lower overall levels: 60% of the tumor cells were lysed in the presence of CAR transduced T cells in the first and second round of coculture. In contrast only 40% of the tumor cells were lysed in the presence of not transduced CAR T cells in the first round and no killing was detectable in the second round. No specific killing was detectable in the first round for E:T ratios of 0.15:1 when not transduced T cells are compared to CAR transduced T cells. In summary, the functionality of CAR transduced T cells manufactured within 3 days was confirmed in vitro as less tumor cells were present after 2 consecutive rounds of coculture were detectable when compared to the not-transduced control.

FIG. 18: In vivo function of CAR T cells generated within 3 days

The in vivo functionality of CAR transduced T cells generated within 3 days was confirmed in 6 to 8 week old NOD scid gamma (NSG) (NOD.Cg-Prkdc^{scid}I12rg^{tm1Wjl}/SzJ) mice. All experiments were performed in compliance with the “Directive 2010/63/EU of the European Parliament and of the Council of 22 Sep. 2010 on the protection of animals used for scientific purposes” and in compliance with the regulations of the German animal protection law.

Briefly, a leukapheresis sample of a healthy donor was automatically processed in the CliniMACS® Prodigy system to generate CAR T cells within 3 days (see FIG. 18 top). On day 0, a bag containing the leukapheresis sample was sterile connected to the CliniMACS Prodigy® Tubing Set 520 by welding. The cells were automatically washed and labelled with CD4 and CD8 CliniMACS reagent to enrich T cells. 2e8 T cells were transferred in IL-7/IL-15 containing medium to the cultivation chamber and were polyclonally stimulated with MACS® GMP T Cell TransAct™ (Miltenyi Biotec) in a cultivation volume of 200 ml. On day 1, the isolated and stimulated T cells were genetically modified with VSV-G pseudotyped lentiviral vectors to induce the expression of CD22/CD19 Tandem-CAR. A bag containing 10 ml of lentiviral vectors was sterile connected to the tubing set and automatically transferred to the chamber containing the T cells. On day 2, 10 ml of a solution containing dextranase were sterile connected to the tubing set and automatically added to the chamber containing the T cells to specifically degrade the linker, thereby the antibodies or fragments specific for CD3 and CD28 are released and the activity of the modulatory agent is inhibited. After washing multiple times the cell product was analyzed by flow cytometry to determine the transduction efficiency, viability and cellular composition at each step (see FIG. 19). Per mouse 3e6 or 6e6 total T cells from CAR transduced groups were injected at the harvesting day (see FIG. 18 bottom). 4d days earlier tumors have been established by intravenously inoculation with 5e5 Firefly luciferase-expressing Raji cells (see FIG. 17). Per group 7 mice were treated. Two additional groups were established as negative control: one group received tumor cells but no T cells (n=7; tumor only) and one group received tumor cells and 3e6 untransduced T cells (n=7) from the same donor cultivated in parallel in small scale. Tumor growth as well as antitumoral response was monitored frequently using an In vivo Imaging System (IVIS Lumina III). For this purpose, 100 μl XenoLight Rediject D-Luciferin Ultra was injected i.p. and subsequently mice were anesthetized using the Isofluran XGI-8 Anesthesia System. Measurement was performed six min after substrate injection. At the end of the experiment spleen, bone marrow and blood was prepared and analyzed by flow cytometry to the determine the frequency of tumor cells and T cell subsets.

FIG. 19: Cellular composition

The cellular composition was determined by flow cytometry by staining for CD45h, CD3, CD4, CD8, CD16/CD56, 7-AAD, CD19, CD14 on samples taken pre enrichment, post enrichment and after harvesting to determine the quality of the cell product. The cellular composition after formulation was 67% CD4 T cells, 18% CD8 T cells and 7% NKT cells. The frequency of NK cells, eosinophils, neutrophils, B cells or monocytes was at background levels confirming the T cell purity after enrichment.

FIG. 20: Representative In vivo imaging data for selected groups

The tumor burden as well as the antitumoral activity of the CAR T cells was monitored frequently by in vivo imaging. All mice are shown for the cohorts containing mice that have received 3e6 viable T cells: Transduced and not transduced. 3 representative mice out of 7 are shown for the tumor only group. The tumor burden increased rapidly for the mice in cohorts that received untransduced T cells or tumor cells only. Mice in both control groups had to be sacrificed 14d post T cell injection as critical levels of tumor burden were reached. In contrast, mice that have received CAR transduced T cells showed a decelerated increase at early time points in an dose-dependent manner 3 and 7 days post T cell injection when compared to the control groups. The level of tumor burden for the CAR transduced T cell groups peaked on day 7 post T cell injection followed by a steady reduction of the tumor burden down to levels measured at the beginning of the experiment.

FIG. 21: In vivo imaging data for all groups

The mean tumor burden +/−SEM measured as p/s over time is shown for all mice for all groups. The data for the 6E6 CAR transduced T cell group (n=7) is included. Mice treated with 6E6 T cells showed a quicker antitumoral response than the 3E6 group. On day 14 post T cell injection the tumor burden was substantially decreased to a comparable, low level for both T cells doses. The control groups (i.e. tumor only and untransduced T cells) were not able to control the tumor growth and mediate potent antitumoral activity.

FIG. 22: Abundance of T cells in bone marrow

The abundance of human T cells in the bone marrow was quantified by flow cytometry for 3 randomly selected mice upon staining for CD45h, CD4, CD8, CD20, CD22, 7-AAD, CD19 CAR Detection (all Miltenyi Biotec). The number of each mouse is shown. For the control groups the analysis was performed on bone marrow sampled on day 14. For the 3e6 CAR transduced T cells group, 3 randomly selected mice were analyzed on day 18. As expected no T cells were found in the Tumor only group. Up to 20% T cells were detectable for the non-transduced cohort. In contrast, the frequency of human T cells was highest with up to 75% in the cohort containing mice that were infused with CAR transduced T cells indicating homing of the CAR T cells to this niche and in vivo proliferation.

FIG. 23: Abundance of tumor and T cells in bone marrow

The human cellular compartment was investigated in more detail to determine the frequency of the human Raji tumor cells and the human T cells. Therefore, the frequency of all human cells was set to 100%. The number of each mouse is shown. As expected no human T cells but only Raji cells were found in the tumor only cohort. Bone marrow is the preferred niche of the Raji tumor cells. In contrast only a minor fraction of Raji cells was detectable in this organ for the CAR transduced T cell group. This is in line with about 50% human CD4 and ˜50% human CD8 T cells present in the organ of these representative mice. 20-60% of the human cells were Raji cells for the untransduced T cell group with a CD4 to CD8 T cell ratio of 2:1 to 3:1.

FIG. 24: Abundance of T cell subsets in spleen

The abundance of T cells in the spleen of 3 randomly selected mice was quantified by flow cytometry upon staining for CD45h, CD4, CD8, CD20, CD22, 7-AAD, CD19 CAR Detection (all Miltenyi Biotec). The number of each mouse is shown. For the control groups the analysis was performed on spleen sampled on day 14. For the 3e6 CAR transduced T cell group, 3 randomly selected mice were analyzed on day 18. As expected no T cells were found in the tumor only group. Up to 10% T cells were detectable for non-transduced cohort. In contrast, the frequency of human T cells was highest with up to 40% in the cohort containing mice that were infused with CAR transduced T cells.

FIG. 25: Abundance of T cell subsets in blood

The abundance of T cells circulating in the blood of 3 randomly selected mice was quantified by flow cytometry upon staining for CD45h, CD4, CD8, CD20, CD22, 7-AAD, CD19 CAR Detection (all Miltenyi Biotec). The number of each mouse is shown. For the control groups the analysis was performed on spleen sampled on day 14. For the 3e6 CAR transduced T cell group, 3 randomly selected mice were analyzed on day 18. No T cells were found in the tumor only group and only minor fractions in the cohort containing mice with untransduced T cells. In contrast, the frequency of human T cells circulating in blood was highest with up to 25% in the cohort containing mice that were infused with CAR transduced T cells.

### DETAILED DESCRIPTION OF THE INVENTION

It is an aspect of the present invention that it provides a method for the generation of genetically modified T cells comprising the steps
a) a sample provided, said sample comprising T cells
b) preparation of said sample by centrifugation
c) enrichment of the T cells of step b (enrichment of the T cells from the prepared sample)
d) activation of the enriched T cells using modulatory agents
e) genetic modification of the activated T cells by transduction with lentiviral vector particles
f) removal of said modulatory agents,

thereby generating a sample of genetically modified T cells,

wherein said method is performed in equal or less than 144 hours, less than 120 hours, less than 96 hours, less than 72 hours, less than 48 hours, or less than 24 hours.

To date, the most prevalent adverse effect following infusion of CAR T cells is the onset of immune activation, known as cytokine release syndrome (CRS). It is a systemic inflammatory response caused by cytokines released by infused CAR T cells shortly after infusion recognizing a potentially high load of tumor cells expressing the CAR antigen. CAR T cell manufacturing within a short period of time may at least partially reduce this toxicity because not all CAR T cell express the CAR at this early time point followed by a steady but slow increase of CAR expression levels (see Example 10).

The combination e.g. of removal of modulatory agents and/or magnetic particles used for enrichment of T cells as disclosed herein and the performance of the method as disclosed herein in equal or less than 144 hours, less than 120 hours, less than 96 hours, less than 72 hours (3 days), less than 48 hours, or less than 24 hours allows successfully to apply to treat i-vivo a patient suffering from e.g. a cancer, wherein the number of T cells in said generated sample of said method is less than 10-fold or less that 5-fold higher compared to the number of T cells in said provided sample.

A sample provided (or providing a sample) comprising T cells may be provided from a subject such as a human (a sample comprising T cells provided by a subject). Said provided sample may be whole blood of a human, a leukapheresis of a subject, buffy coat, PBMC, outgrown or isolated T cells.

Preparation of said sample may result in volume reduction, rebuffering, removal of serum, erythrocyte reduction, platelet removal, and/or washing.

Alternatively said method may start with step a: providing a sample comprising T cell.

Alternatively said method may start with step b: preparation of a sample comprising T cells by centrifugation. This alternative step b may be followed by steps c to f.

Said method, wherein said sample of step a) comprises human serum and wherein said serum is removed by step b).

Said human serum may comprise components that reduce the transduction efficiency of the lentiviral vector particle into the cell. Said components of the human serum that may reduce said transduction efficiency may be components of the complement system of a subject or may be neutralizing antibodies (see e.g. DePolo et al, 2000, Molecular Therapy, 2: 218-222). The removal of human serum may be performed by washing (a washing step) achieved by said centrifugation. The washing step may be performed by a series of media/buffer exchanges (at least twice exchanges) thereby removing the human serum and/or its components from the T cells. Said method, wherein said T cell are prepared and enriched in less than 2 hours, preferentially in less than 1 hour.

Said method, wherein said T cells are activated (stimulated) using said modulatory agents in less than 72 hours, preferentially in less than 48 hours, more preferentially in less than 24 hours, i.e. the addition of said modulatory agents and the removal of said modulatory agents occur within the period of said hours.

Said method, wherein the transduction of said activated T cells starts 2 days after said stimulation of T cells using modulatory agents, preferentially 1 day after said stimulation, more preferentially at the same time as said stimulation.

Said method, wherein said modulatory activating agents may be removed (step f) in less than 2 hours, preferentially in less than 1 hour, more preferentially in less than 30 minutes after the addition of said modulatory agents to the T cells (step d).

Said method, wherein said genetic modification of said T cells by transduction with lentiviral vector particles (step e) may be performed in less than 2 days, preferentially in less than 1 day, more preferentially in less than 12 hours.

Said method, wherein the T cells of the provided sample may be enriched prior to said genetic modification of the T cells for CD4 positive and/or CD8 positive T cells by using CD4 and/or CD8 as positive selection marker, and/or wherein the T cells of the provide sample may be depleted of cancer cells that contaminate the sample comprising T cells by using a tumor associated antigen (TAA) as a negative selection marker. The TAA may be selected from one or more markers of e.g. CD19, CD20, CD22, CD30, CD33, CD70, IgK, IL-1Rap, Lewis-Y, NKG2D ligands, ROR1, CAIX, CD133, CEA, c-MET, EGFR, EGFRvIII, EpCam, EphA2, ErbB2/Her2, FAP, FR-a, GD2, GPC3, IL-13Ra2, L1-CAM, Mesothelin, MUC1, PD-L1, PSCA, PSMA, VEGFR-2, BCMA, CD123 and CD16V.

Said enrichment of CD4+ and/or CD8+ T cells and/or depletion of cancer cells from the provided sample may be performed by a separation step. Said separation may be performed by flow cytometry methods (fluorescence activated cell sorting) such as FACSorting, magnetic cell separation such as MACS or by microchip based cell sorting such as MACSQuant® Tyto®. Preferred is the use of a magnetic cell separation step.

Said method, wherein said enrichment of CD4 and/or CD8 positive T cells is performed by magnetic cell separation steps comprising:

i) contacting the T cells with magnetic particles that are directly or indirectly coupled to antibodies or antigen binding fragments thereof specific for CD4 and/or CD8, wherein said magnetic particles and said antibodies or antigen binding fragments thereof coupled thereto can be removed

ii) separating the CD4 and/or CD8 T cells in a magnetic field

iii) removal of said magnetic particles from the enriched T cells after the separation.

Said method, wherein said enrichment of CD4 and/or CD8 positive T cells is performed by magnetic cell separation steps comprising:

i) contacting the T cells with magnetic particles that are directly or indirectly coupled to antibodies or antigen binding fragments thereof specific for CD4 and/or CD8, wherein said magnetic particles and said antibodies or antigen binding fragments thereof coupled thereto can be removed by washing

ii) separating the CD4 and/or CD8 T cells in a magnetic field

iii) removal of said magnetic particles from the enriched T cells after the separation step by washing.

Said method, wherein said enrichment of CD4 and/or CD8 positive T cells is performed by magnetic cell separation steps comprising:

i) contacting the T cells with magnetic particles that are directly or indirectly coupled to antibodies or antigen binding fragments thereof specific for CD4 and/or CD8, wherein said magnetic particles and said antibodies or antigen binding fragments thereof coupled thereto can be disrupted chemically and/or enzymatically

ii) separating the CD4 and/or CD8 T cells in a magnetic field

iii) removal of said magnetic particles from the enriched T cells after the separation step by chemical and/or enzymatical disruption of said magnetic particles and said antibodies or antigen binding fragments thereof coupled thereto.

Said removal of said magnetic particles from the enriched T cells after the separation step by chemical and/or enzymatical disruption may be performed within the magnetic field or after removal of the magnetic field.

Methods and systems for removal of magnetic particles from a cell that have been directly or indirectly bound to said cell are well-known in the art.

Exemplary, some methods and systems for reversible labelling of a cell with magnetic particles that lead to a disruption of magnetic particles from the cells are listed here.

One strategy exploits the specific competition of a non-covalent binding interaction. US20080255004 discloses a method for reversible binding to a solid support, e.g., magnetic particle, using antibodies recognizing the target moiety which are conjugated to modified biotin like desthiobiotin, and modified streptavidin or avidin bound to the solid support. The binding interaction of the modified binding partners is weaker compared to the strong and specific binding between biotin and streptavidin therefore facilitating the dissociation in the presence of these competitors. EP2725359B1 describes a system for reversible magnetic cell separation based on the non-covalent interaction of a ligand-PEO-Biotin-conjugate recognizing the target moiety and an anti-Biotin-antibody compromising a magnetic particle that can be released by adding the competing molecule biotin, streptavidin or an auxiliary reagent.

Said method, wherein said enrichment of CD4 and/or CD8 positive T cells is performed by magnetic cell separation step comprising:

i) contacting the T cells with magnetic particles that are indirectly coupled via a linker to antibodies or antigen binding fragments thereof specific for CD4 and/or CD8, wherein said magnetic particles and said antibodies or antigen binding fragments thereof coupled thereto can be removed by adding a competing agent that competes with the binding of said linker to said antibodies or antigen binding fragments thereof

ii) separating the CD4 and/or CD8 T cells in a magnetic field

iii) removal of said magnetic particles from the enriched T cells after the separation step by adding the competing agent.

Said method, wherein said competing agent is biotin, streptavidin or an auxiliary reagent.

Beside these competitive release mechanisms, the removal of labelling is mentioned by mechanical agitation, chemically cleavable or enzymatically degradable linkers. WO 96/31776 describes a method to release after separation magnetic particles from target cells by enzymatically cleaving a moiety of the particle coating, or a moiety present in the linkage group between the coating and the antigen recognizing moiety. An example is the application of magnetic particles coated with dextran and/or linked via dextran to the antigen recognizing moiety. Subsequent cleavage of the isolated target cells from the magnetic particle is initiated by the addition of the dextran-degrading enzyme dextranase. A related method in EP3037821 discloses the detection and separation of a target moiety according to, e.g. a fluorescence signal, with conjugates having an enzymatically-degradable spacer.

Recently, the interest grew in techniques utilizing antigen recognizing moieties whose binding to the target moiety is characterized by a low-affinity constant. To ensure a specific and stable labelling with those low-affinity antigen recognizing moieties the structure of the labelling conjugate has to comprise a multimerization of the antigen recognizing moiety providing high avidity. Upon disruption of the multimerization the low-affinity antigen recognizing moiety can dissociate from the target moiety therefore providing the opportunity to release at its best the detection moiety and the antigen recognizing moiety from the target moiety.

This reversible multimer staining was first described in U.S. Pat. No. 7,776,562 respectively U.S. Pat. No. 8,298,782 wherein the multimerization is build up by a non-covalent binding interaction. Exemplary, low affinity peptide/MHC-monomers having a StreptagII are multimerized with streptactin and the multimerization is reversible upon addition of the competing molecule biotin.

The method was revised in U.S. Pat. No. 9,023,604 regarding the characteristics of the antigen recognizing moiety respectively receptor binding reagent to enable reversible labelling. Receptor binding reagents characterized by a dissociation rate constant about 0.5×10−4 sec-1 or greater with a binding partner C are multimerized by a multimerization reagent with at least two binding sites Z interacting reversibly, non-covalently with the binding partner C to provide complexes with high avidity for the target antigen. The detectable label is bound to the multivalent binding complex. Reversibility of multimerization is initiated upon disruption of the binding between binding partner C and the binding site Z of the multimerization reagent. For example, in multimers of Fab-StreptagII/Streptactin, multimerization can be reversed by the competitor Biotin.

In EP0819250B1 a method is provided for releasing magnetic particles bound to a cell surface through an affinity reagent, e.g. an antibody or antigen binding fragment thereof. The magnetic particle is released through action of a glycosidase specific for a glycosidic linkage present in at least one of (a) the coating of the particle and (b) a linkage group between the coating and the affinity reagent.

In EP3336546A1 a method is disclosed for detecting a target moiety in a sample of biological specimens by:
a) providing at least one conjugate with the general formula (I)

Aₙ−P−Bₘ−C_{q}−Xₒ (I)
with A: antigen recognizing moiety;
   P: enzymatically degradable spacer;
   B: first binding moiety
   C second binding moiety
   X: detection moiety;
   n, m, q, o integers between 1 and 100,
   wherein B and C are non-covalently bound to each other and A and B are covalently bound to P
b) labelling the target moiety recognized by the antigen recognizing moiety A with at least one conjugate
c) detecting the labelled target moiety via detecting moiety X
d) cleaving C_{q}-Xₒ by disrupting the non-covalent bond between Bₘ and C_{q} from the labelled target moiety
e) cleaving the binding moiety Bₘ from the labelled target moiety by enzymatically degrading spacer P.

The method of EP3336546A1 may be utilized not only for detecting target moieties i.e. target cells expressing such target moieties, but also for isolating the target cells from a sample of biological specimens. The isolating procedures makes use of detecting the target moieties. For example, the detection of a target moiety by fluorescence may be used to trigger an appropriate separation process as performed on FACS or TYTO separation systems. In the method in EP3336546A1, the well-known magnetic cell separation process can also be used as detection and separation process, wherein the magnetic particles are detected by the magnetic field.

In a preferred embodiment of the invention, said magnetic particles that are directly coupled to antibodies or antigen binding fragments thereof specific for CD4 and/or CD8 are coupled via a biodegradable linker, wherein said biodegradable linker is degraded by adding an enzyme that (specifically) digests the biodegradable linker. Said biodegradable linker may be or may comprise a polysaccharide and said enzyme that specifically digests the glycosidic linkages is a hydrolase. Said biodegradable linker may be or may comprise dextran and said enzyme that (specifically) digests dextran may be dextranase.

In another preferred embodiment of the invention, said magnetic particles that are indirectly coupled to antibodies or antigen binding fragments thereof, such as Fabs, specific for CD4 and/or CD8 are coupled via two components

i) a linker, such as dextran, that is coupled to a tag such as PEO-Biotin or said Fabs specific for CD4 and/or CD8 that are coupled to a tag such as PEO-biotin,

ii) a magnetic particle that is coupled to an antibody or antigen binding fragment thereof specific for said tag, e.g. biotin, wherein after combining component i and ii and after contacting the T cells with said indirectly coupled magnetic particle, the magnetic particle may be disrupted (removed) by adding a competing agent that competes with said tag, e.g. biotin (as a competitor).

In another preferred embodiment of the invention, said magnetic particles that are indirectly coupled to antibodies or antigen binding fragments thereof, such as Fabs, specific for CD4 and/or CD8 are coupled via two components

i) a biodegradable linker, such as dextran, that is coupled to a tag such as PEO-Biotin,

ii) a magnetic particle that is coupled to an antibody or antigen binding fragment thereof specific for said tag, e.g. biotin, wherein after combining component i and ii and after contacting the T cells with said indirectly coupled magnetic particle, the magnetic particle may be disrupted from said T cell by adding an enzyme that specifically digests the biodegradable linker such as dextranase and/or by adding a competing agent that competes with said tag, e.g. biotin (as a competitor). Said method, wherein said competing agent is biotin, streptavidin or an auxiliary reagent. The principle of this embodiment of the invention is illustrated with regard to the release/disruption principle in the FIG. 4.

Said method, wherein said modulatory agents comprise an antibody or antigen binding fragment thereof specific for CD3 and/or an antibody or antigen binding fragment thereof specific for CD28 coupled directly or indirectly via a linker, wherein said antibodies or antigen binding fragments thereof specific for CD3 and CD28 can be removed.

Said removal of the modulatory agents from the cells may be further performed by one or more washing steps.

Said method, wherein said modulatory agents comprise an antibody or antigen binding fragment thereof specific for CD3 and/or an antibody or antigen binding fragment thereof specific for CD28 coupled directly or indirectly via a linker, wherein said antibodies or antigen binding fragments thereof specific for CD3 and CD28 can be disrupted chemically and/or enzymatically, and wherein said modulatory agents are removed by chemical and/or enzymatical disruption of said antibodies or antigen binding fragments thereof specific for CD3 and CD28. Removal of the disrupted modulatory agents from the cells may be further performed by one or more washing steps.

The methods and systems described above for removal of magnetic particles from a cell that have been directly or indirectly bound to said cell may also be suitable, may be transferred to and/or may be applied for the removal of said modulatory agents that comprise an antibody or antigen binding fragment thereof specific for CD3 and an antibody or antigen binding fragment thereof specific for CD28 coupled directly or indirectly via a linker.

Said method, wherein said removal of said modulatory agents of said antibodies or antigen binding fragments thereof specific for CD3 and/or CD28 is performed by

a) a competitive reaction comprising the step of adding a competing agent that competes with a tag, e.g. biotin (as a competitor), if said modulatory agents comprise indirectly coupled antibodies or antigen binding fragments thereof, such as Fabs, specific for CD3 and/or CD28 via two components, wherein said two components may be

i) antibodies or antigen binding fragments thereof, such as Fabs, specific for CD3 and/or CD28 are coupled to said tag such as PEO-Biotin, or antibodies or antigen binding fragments thereof, such as Fabs, specific for CD3 and/or CD28 that are coupled via a linker such as dextran that is coupled to said tag such as PEO-biotin, and

ii) antibodies or antigen binding fragments thereof, such as Fabs, specific for the tag, e.g. biotin, and wherein said to components i) and ii) have been combined and contacted with said cells, and/or

b) an enzymatic disruption comprising the step of adding an enzyme that biodegrades said linker, if the linker is a biodegradable linker (i.e. an indirect or direct linkage of the two antibodies or antigen binding fragments thereof via the linker).

In another preferred embodiment of the invention, said modulatory agents comprise indirectly coupled antibodies or antigen binding fragments thereof, such as Fabs, specific for CD3 and/or CD28 via two components:

i) antibodies or antigen binding fragments thereof, such as Fabs, specific for CD3 and/or CD28 are coupled to a tag such as PEO-Biotin, or antibodies or antigen binding fragments thereof, such as Fabs, specific for CD3 and/or CD28 that are coupled via a linker such as dextran that is coupled to a tag such as PEO-biotin

ii) antibodies or antigen binding fragments thereof, such as Fabs, specific for the tag, e.g. biotin, wherein after combining component I and ii and after contacting the T cells with said combined components, said combined components may be disrupted (removed) by adding a competing agent that competes with said tag, e.g. biotin (as a competitor).

Said biodegradable linker may be or may comprise a polysaccharide and said enzyme that specifically digests the glycosidic linkages may be a hydrolase.

Said biodegradable linker may be or may comprise dextran and said enzyme that specifically digests dextran may be dextranase.

In a preferred embodiment of the invention, said modulatory agents comprise an antibody or antigen binding fragment thereof specific for CD3 and/or an antibody or antigen binding fragment thereof specific for CD28 that are directly coupled via a biodegradable linker, wherein said biodegradable linker is degraded by adding an enzyme that specifically digests the biodegradable linker. Said biodegradable linker may be or may comprise a polysaccharide and said enzyme that specifically digests the glycosidic linkages is a Hydrolase.

Said biodegradable linker may be or may comprise dextran and said enzyme that specifically digests dextran may be dextranase.

Said method, wherein after the genetic modification of the T cells by transduction with lentiviral vector particles residual lentiviral vector particles are removed.

Said removal of residual lentiviral vector particles may be performed before, subsequent or after the removal of said modulatory agents and/or said removal of said magnetic particles.

Said removal of residual lentiviral vector particles may be performed by washing, wherein the washing results in an at least 10-fold, preferably 100-fold reduction of residual vector particles in the sample that comprises the genetically modified T cells.

The washing step may be performed by a series of media/buffer exchanges (at least twice exchanges) thereby removing said residual lentiviral vector particles from said sample comprising said genetically modified T cells. The exchanges may be performed by separation of cells and media/buffer by centrifugation, sedimentation, adherence or filtration and subsequent exchange of media/buffer.

The at least 10-fold, preferably 100-fold reduction of residual vector particles in the sample that comprises the genetically modified T cells by washing can be achieved for example by
i) Separating cells and media/buffer
ii) Removal of 90%, preferably 99% of the volume of media/buffer
iii) Adding new media/buffer to the original volume.
iv) Resuspension of cells in media/buffer

Washing steps may be performed in a consecutive manner that may result in a cumulative reduction of lentiviral vectors (i.e. two washing steps with a 10-fold reduction per step result in cumulative reduction of 100-fold).

Said removal of residual lentiviral vector particles may be performed by incubation with substances that inactivate lentiviral vector particles and/or reduce their stability. Substances that inactivate lentiviral vector particles and/or reduce their stability may be washed away after said incubation, wherein said incubation occurs for no longer than 3 hours, preferentially no longer than 1 hour.

Such substances that inactivate lentiviral vector particles and/or reduce their stability may be e.g. Heparin, antiretrovirals, complement factors of a human blood, neutralizing antibodies that a contained in human blood or a mild basic buffer.

Said antiretrovirals may be e.g. inhibitors of viral enzymes such as Zidovudin (zidothymidin, AZT) or Raltegravir.

Complement factors and/or neutralizing antibodies that are contained in blood, e.g. human blood, may be isolated by methods well-known in the art.

The mild basic buffer may have a pH value of about 7 to 9, being sufficiently mild to not harm the T cells of the sample. Such a buffer is described e.g in Holic et al. (Hum Gene Ther Clin Dev. 2014 September; 25(3):178-85)

Said method, wherein the removed human serum as disclosed herein or isolated substances therefrom such as complement factors and/or neutralizing antibodies that inhibit productive transduction of lentiviral vector particles to T cells may be added to the genetic modified T cells, thereby removing and/or neutralizing residual lentiviral vector particles.

The method as disclosed herein, wherein said method is an automated method, preferentially performed in a closed system.

The method as disclosed herein can be fully implemented as an automated process, preferentially in a closed system under GMP conditions.

Such a closed system allows to operate under GMP or GMP-like conditions (“sterile”) resulting in cell compositions which are clinically applicable. Herein exemplarily the CliniMACS Prodigy® (Miltenyi Biotec GmbH, Germany) is used as a closed system. This system is disclosed in WO2009/072003. But it is not intended to limit the use of the method of the present invention to the CliniMACS® Prodigy.

The CliniMACS Prodigy® System is designed to automate and standardize complete cellular product manufacturing processes. It combines CliniMACS® Separation Technology (Miltenyi Biotec GmbH, Germany) with a wide range of sensor-controlled, cell processing capabilities. Prominent features of the device are:
disposable CentriCult™ Chamber enabling standardized cell processing and cultivation
Cell enrichment and depletion capabilities, alone or combined with CliniMACS® Reagents (Miltenyi Biotec GmbH)
Cell cultivation and cell expansion capabilities thanks to temperature and controlled CO2 gas exchange.
Final product formulation in pre-defined medium and volume
the possibility to program the device using Flexible Programming Suite (FPS) and GAMP5 compatible programming language for customization of cell processing
Tailor-made tubing sets for a variety of applications

The centrifugation chamber and the cultivation chamber may be identical. The centrifugation chamber and the cultivation chamber can be used in various conditions: for example, for separation or transduction, high rotational speed (i.e. high g-forces) can be applied, whereas for example, culturing steps may be performed with slow rotation or even at idle state. In another variant of the invention, the chamber changes direction of rotation in an oscillating manner that results in a shaking of the chamber and maintenance of the cell in suspension. Accordingly, in the process of the invention, T cell stimulation, gene modifying and/or cultivation steps can be performed under steady or shaking conditions of the centrifugation or the cultivation chamber.

Said method, wherein the number of T cells in the generated sample may be less than 10-fold, preferentially less than 5-fold higher compared to the number of T cells in said provided sample.

Said method, wherein the generated T cells underwent less than 4, preferentially less than 3 cell divisions.

There is no need to expand in-vitro the engineered T cells to cell numbers that have been known to be required for effective treatment in a patient as the further expansion of these genetically T cells to therapeutic effective amounts of cells will take place in vivo (see e.g. Ghassemi et al, 2018, Cancer Immunol Res 6:1100-1109). This is possible due to the high quality of composition/sample of genetically modified T cells generated by the method as disclosed herein, i.e. the low contamination with non-engineered T cell components and toxic substances.

The omission of in-vitro expanding/proliferation of the genetically modified T cells to larger cell numbers allows for a reduction of time needed to prepare a clinical applicable composition comprising modified T cells.

Said genetically modified T cells may be genetically modified to express a chimeric antigen receptor (CAR), a T cell receptor (TCR), or any accessory molecule, on their cell surface.

For final formulation, the genetically modified T cells may be washed by centrifugation and replacement of culture medium with a buffer appropriate for subsequent applications such as infusion of the generated cell composition into a patient.

When required, genetically-modified T cells can be separated from non-modified T cells e.g. using again the magnetic separation technology.

In one aspect the present invention provides a cell composition obtained by the methods as disclosed herein.

In one embodiment of the invention said cell composition is a pharmaceutical cell composition optionally comprising a pharmaceutical carrier.

The method of the present invention may comprise any embodiment of the invention and/or step as described herein in any order and/or combination resulting in a functional method for the generation of genetically modified T cells as disclosed herein.

In addition to above described applications and embodiments of the invention further embodiments of the invention are described in the following without intention to be limited to these embodiments.

### EMBODIMENTS

In a preferred embodiment of the invention, T cells are genetically modified in a closed system in an automated process, e.g. by using the CliniMACS® Prodigy (Miltenyi Biotec GmbH) to express a chimeric antigen receptor.

A sample comprising T cells may be provided that originate from a human e g suffering from cancer. The human serum of the provided sample comprising T cells may be washed away by a centrifugation step.

CD4+ and/or CD8+ T cells may be enriched by a magnetic separation step using anti-CD4 and/or anti-CD8 antibodies or antigen binding fragments thereof coupled via dextran to a magnetic particle. After separation of CD4+ and/or CD8+ T cells in a magnetic field from the sample comprising T cells the magnetic particle is removed from the enriched cells by adding dextranase that disrupt the binding of the antibodies or fragments thereof to the magnetic particle by cleavage of the dextran chains.

The enriched CD4+ and/or CD8+ T cells may be activated for 24 hours using an antibody or antigen binding fragment thereof specific for CD3 and an antibody or antigen binding fragment thereof specific for CD28 coupled via a linker that comprises dextran as a modulatory agent.

Lentiviral vector particles that comprise nucleic acid that encodes for a CAR may be added the sample comprising activated CD4+ and/or CD8+ T cells. Transduction may be performed during the stimulation or after the stimulation for 24 hours.

After transduction of the lentiviral particles into the CD4+ and/or CD8+ T cells the modulatory agent is washed away or removed by adding dextranase that disrupt the binding of the antibodies or fragments thereof to each other by cleavage of the dextran chains. Residual lentiviral vector particles are reduced in the sample comprising genetically modified T cells at least 10-fold, preferentially at least 100-fold by repeated washing. As a result a pure sample comprising genetically modified T cells is achieved in equal or less than 144 hours, less than 120 hours, less than 96 hours, less than 72 hours, less than 48 hours, or less than 24 hours, and the expansion of the genetically modified T cells in the generated sample is less than 10-fold, preferentially less than 5-fold compared to the amount of T cells of the originally provided sample comprising T cells. The sample or composition comprising the genetically modified T cells may be applied to said human and said genetically modified T cells may express a CAR that recognizes an TAA in said human.

### Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

The terms “modulatory agents”, “activating agents” and “stimulating agents” as used herein may be used interchangeably.

The modulatory agents may be selected from the group consisting of agonistic antibodies or antigen binding fragment thereof, cytokines, recombinant costimulatory molecules and small drug inhibitors. Said modulatory agents are anti-CD3 and anti-CD28 antibodies or fragments thereof coupled to beads or nanostructures. The modulatory agents may be a nanomatrix, the nanomatrix comprising a) a matrix of mobile polymer chains, and b) attached to said matrix of mobile polymer chains anti-CD3 and anti-CD28 antibodies or fragments thereof, wherein the nanomatrix is 1 to 500 nm in size. The anti-CD3 and anti-CD28 antibodies or fragments thereof may be attached to the same or to separate matrices of mobile polymer chains. If the anti-CD3 and anti-CD28 antibodies or fragments thereof are attached to separate matrices of mobile polymer chains, fine-tuning of nanomatrices for the stimulation of the T cells is possible. The nanomatrix may be biodegradable. The nanomatrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. The choice of degrading enzyme agent will be determined by the glycosidic linkage. Where the macromolecular coating is a polysaccharide, the polysaccharide will be chosen to have glycosidic linkages not normally found in mammalian cells. Hydrolases that recognize specific glycosidic structures may be used as an enzyme e.g. dextran and dextranase, which cleaves at the α(1→6) linkage; cellulose and cellulase, which cleaves at the μ(1→4) linkage; amylose and amylase; pectin and pectinase; chitin and chitinase, etc.

In addition sterile filtration of said small nanomatrices as disclosed e.g. in WO2014/048920A1 is possible which is an important feature for T cell activation under conditions which are compliant with rigorous GMP standards, i.e. in a closed system.

The term “depletion” as used herein refers to a process of a negative selection that separates the desired cells from the undesired cells, herein normally the cancer cells, which are labelled by an antibody or antigen-binding fragment thereof coupled to a solid phase such as a particle, fluorophore or hapten.

The term “particle” as used herein refers to a solid phase such as colloidal particles, microspheres, nanoparticles, or beads. Methods for generation of such particles are well known in the field of the art. The particles may be magnetic particles. The particles may be in a solution or suspension or they may be in a lyophilised state prior to use in the present invention. The lyophilized particle is then reconstituted in convenient buffer before contacting the sample to be processed regarding the present invention.

The term “magnetic” in “magnetic particle” as used herein refers to all subtypes of magnetic particles which can be prepared with methods well known to the skilled person in the art, especially ferromagnetic particles, superparamagnetic particles and paramagnetic particles.

“Ferromagnetic” materials are strongly susceptible to magnetic fields and are capable of retaining magnetic properties when the field is removed. “Paramagnetic” materials have only a weak magnetic susceptibility and when the field is removed quickly lose their weak magnetism. “Superparamagnetic” materials are highly magnetically susceptible, i.e. they become strongly magnetic when placed in a magnetic field, but, like paramagnetic materials, rapidly lose their magnetism.

The linkage between antibody (or an antigen binding fragment thereof) and particle can be covalent or non-covalent. A covalent linkage can be, e.g. the linkage to carboxyl-groups on polystyrene beads, or to NH₂ or SH₂ groups on modified beads. A non-covalent linkage is e.g. via biotin-avidin or a fluorophore-coupled-particle linked to anti-fluorophore antibody. Methods for coupling antibodies to particles, fluorophores, haptens like biotin or larger surfaces such as culture dishes are well known to the skilled person in the art.

For enrichment, isolation or selection in principle any sorting technology can be used. This includes for example affinity chromatography or any other antibody-dependent separation technique known in the art. Any ligand-dependent separation technique known in the art may be used in conjunction with both positive and negative separation techniques that rely on the physical properties of the cells. An especially potent sorting technology is magnetic cell sorting. Methods to separate cells magnetically are commercially available e.g. from Invitrogen, Stem cell Technologies, in Cellpro, Seattle or Advanced Magnetics, Boston. For example, monoclonal antibodies can be directly coupled to magnetic polystyrene particles like Dynal M 450 or similar magnetic particles and used e.g. for cell separation. The Dynabeads technology is not column based, instead these magnetic beads with attached cells enjoy liquid phase kinetics in a sample tube, and the cells are isolated by placing the tube on a magnetic rack. However, in a preferred embodiment for enriching CD4+ and/or CD8+ T cells from a sample comprising T cells according the present invention monoclonal antibodies or antigen binding fragments thereof are used in conjunction with colloidal superparamagnetic microparticles having an organic coating by e.g. polysaccharides (Magnetic-activated cell sorting (MACS) technology (Miltenyi Biotec, Bergisch Gladbach, Germany)). These particles (nanobeads or MicroBeads) can be either directly conjugated to monoclonal antibodies or used in combination with anti-immunoglobulin, avidin or anti-hapten-specific MicroBeads.

The MACS technology allows cells to be separated by incubating them with magnetic nanoparticles coated with antibodies directed against a particular surface antigen. This causes the cells expressing this antigen to attach to the magnetic nanoparticles. Afterwards the cell solution is transferred on a column placed in a strong magnetic field. In this step, the cells attach to the nanoparticles (expressing the antigen) and stay on the column, while other cells (not expressing the antigen) flow through. With this method, the cells can be separated positively or negatively with respect to the particular antigen(s)/marker(s).

In case of a positive selection the cells expressing the antigen(s) of interest, which attached to the magnetic column, are washed out to a separate vessel, after removing the column from the magnetic field.

In case of a negative selection the antibody used is directed against surface antigen(s) which are known to be present on cells that are not of interest. After application of the cells/magnetic nanoparticles solution onto the column the cells expressing these antigens bind to the column and the fraction that goes through is collected, as it contains the cells of interest. As these cells are non-labelled by an antibody coupled to nanoparticels, they are “untouched”.

The procedure can be performed using direct magnetic labelling or indirect magnetic labelling. For direct labelling the specific antibody is directly coupled to the magnetic particle. Indirect labelling is a convenient alternative when direct magnetic labelling is not possible or not desired. A primary antibody, a specific monoclonal or polyclonal antibody, a combination of primary antibodies, directed against any cell surface marker can be used for this labelling strategy. The primary antibody can either be unconjugated, biotinylated, or fluorophore-conjugated. The magnetic labelling is then achieved with anti-immunoglobulin MicroBeads, anti-biotin MicroBeads, or anti-fluorophore MicroBeads.

The term “disruption” as used herein in the context of disruption of a magnetic particle or modulatory agent for activation may refer to the removal

by washing alone and/or

by adding a competing agent and subsequent washing and/or

by chemical disruption, i.e. by adding a substance (non-proteinous, chemical compound) that breaks covalent bonds and/or

by enzymatic disruption and subsequent washing, and/or

by input of energy (physical disruption) that breaks covalent bonds.

The term “competitive reaction” in the context of disruption as used herein refers to a magnetic particle or modulatory agent for activation that comprise 2 components, that are not covalently linked wherein one component binds to said cell via antibodies or antigen binding fragments specific for CD3, CD28, CD4 and/or CD28 and contains a tag and a second component that binds to said tag and wherein said binding to so said tag may be dissolved by the addition of a competitor. The competitor may compete and/or replace one component, said magnetic particle, said modulatory agent or said antibodies or antigen binding fragments thereof for example due to higher affinity to the respective component or due to higher concentration of the competitor molecule compared to concentration of the magnetic particle or modulatory agent that is indirectly coupled to antibodies or antigen binding fragments thereof specific for CD3, CD28, CD4 and/or CD8. The term “enzymatical disruption” as used herein in the context of disruption of magnetic particles or modulatory agents refers to antibodies or antigen binding fragments thereof specific for CD3, CD28, CD4 or CD8 that are directly or indirectly linked via a biodegradable linker and wherein said biodegradable linker may be specifically biodegraded, digested or cut by the activity of said enzyme and thereby split said magnetic particle or modulatory agent in at least two separate molecules. Released single antibody or antigen binding fragment thereof such as a Fab specific for CD3 or CD28 then has no further effect on activation of the T cell to which it is bound. In addition, if said antibody or antigen binding fragment thereof such as said Fab has low affinity and/or a high k(off) rate said antibody or antigen binding fragment thereof such as said Fab will be removed from the cell to that is has bound.

The term “chemical disruption” as used herein in the context of disruption of magnetic particles or modulatory agents refers to antibodies or antigen binding fragments thereof specific for CD3, CD28, CD4 or CD8 that are directly or indirectly linked via a chemically degradable linker and wherein said chemically degradable linker may be specifically degraded or cleaved by the addition of a non-proteinous, chemical substance that breaks covalent bonds under physiological conditions and thereby split said magnetic particle or modulatory agent in at least two separate molecules. Examples for suitable reactions for chemical disruption under physiological conditions may be reductions, such as the reduction of disulfide bonds by a reducing agent or the reduction of diazo bonds with dithionite, or oxidations, such as the cleavage of glycol residues by periodate. Released single antibody or antigen binding fragment thereof such as a Fab specific for CD3 or CD28 then has no further effect on activation of the T cell to which it is bound. In addition, if said antibody or antigen binding fragment thereof such as said Fab has low affinity and/or a high k(off) rate said antibody or antigen binding fragment thereof such as said Fab will be removed from the cell to that is has bound.

The term “physical disruption” as used herein in the context of disruption of magnetic particles or modulatory agents refers to antibodies or antigen binding fragments thereof specific for CD3, CD28, CD4 or CD8 that are directly or indirectly linked via a physically disruptable linker and wherein said physically disruptable linker may be specifically degraded or cleaved by energy input that breaks covalent bonds under physiological conditions and thereby split said magnetic particle or modulatory agent in at least two separate molecules. Examples for suitable reactions for physical disruption under physiological conditions may be photo-reactions, such as the photocleavage of light sensitive linkers by UV or visible light as exemplified by the cleavage of ortho-nitrobenzyl derivatives by near-UV light (300-365 nm). Released single antibody or antigen binding fragment thereof such as a Fab specific for CD3 or CD28 then has no further effect on activation of the T cell to which it is bound. In addition, if said antibody or antigen binding fragment thereof such as said Fab has low affinity and/or a high k(off) rate said antibody or antigen binding fragment thereof such as said Fab will be removed from the cell to that is has bound.

The term “marker” as used herein refers to a cell antigen that is specifically expressed by a certain cell type. Preferentially, the marker is a cell surface marker so that enrichment, isolation and/or detection of living cells can be performed. The markers may be positive selection markers such as CD4, CD8 and/or CD62L or may be negative selection markers (e.g. depletion of cells expressing CD14, CD16, CD19, CD25, CD56).

The term “expression” as used herein is defined as the transcription and/or translation of a particular nucleotide sequence driven by its promoter in a cell.

The term “antigen-binding molecule” as used herein refers to any molecule that binds preferably to or is specific for the desired target molecule of the cell, i.e. the antigen. The term “antigen-binding molecule” comprises e.g. an antibody or antigen binding fragment thereof. The term “antibody” as used herein refers to polyclonal or monoclonal antibodies, which can be generated by methods well known to the person skilled in the art. The antibody may be of any species, e.g. murine, rat, sheep, human. For therapeutic purposes, if non-human antigen binding fragments are to be used, these can be humanized by any method known in the art. The antibodies may also be modified antibodies (e.g. oligomers, reduced, oxidized and labeled antibodies).

The term “antibody” comprises both intact molecules and antigen binding fragments, such as Fab, Fab′, F(ab′)2, Fv and single-chain antibodies. Additionally, the term “antigen-binding fragment” includes any molecule other than antibodies or antibody fragments that binds preferentially to the desired target molecule of the cell. Suitable molecules include, without limitation, oligonucleotides known as aptamers that bind to desired target molecules, carbohydrates, lectins or any other antigen binding protein (e.g. receptor-ligand interaction). The linkage (coupling) between antibody and particle or nanostructure can be covalent or non-covalent. A covalent linkage can be, e.g. the linkage to carboxyl-groups on polystyrene beads, or to NH₂ or SH₂ groups on modified beads. A non-covalent linkage is e.g. via biotin-avidin or a fluorophore-coupled-particle linked to anti-fluorophore antibody.

The terms “specifically binds to” or “specific for” with respect to an antigen-binding molecule, e.g. an antibody or fragment thereof, refer to an antigen-binding molecule (in case of an antibody or fragment thereof to an antigen-binding domain) which recognizes and binds to a specific antigen in a sample, e.g. CD4, but does not substantially recognize or bind other antigens in said sample. An antigen-binding domain of an antibody or fragment thereof that binds specifically to an antigen from one species may bind also to that antigen from another species. This cross-species reactivity is not contrary to the definition of “specific for” as used herein. An antigen-binding domain of an antibody or fragment thereof that specifically binds to an antigen, e.g. the CD4 antigen, may also bind substantially to different variants of said antigen (allelic variants, splice variants, isoforms etc.). This cross reactivity is not contrary to the definition of that antigen-binding domain as specific for the antigen, e.g. for CD4.

The terms “genetically modified T cell” or “engineered T cell” may be used interchangeably and mean containing and/or expressing a foreign gene or nucleic acid sequence which in turn modifies the genotype or phenotype of the cell or its progeny. Especially, the terms refer to the fact that cells can be manipulated by recombinant methods well known in the art to express stably or transiently peptides or proteins, e.g. CARs which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons, designer nucleases including zinc finger nucleases, TALENs or CRISPR/Cas.

The genetically modified T cells obtainable by the methods as disclosed herein may be used for subsequent steps such as research, diagnostics, pharmacological or clinical applications known to the person skilled in the art.

The genetically modified T cells may also be used as a pharmaceutical composition in the therapy, e.g. cellular therapy, or prevention of diseases. The pharmaceutical composition may be transplanted into an animal or human, preferentially a human patient. The pharmaceutical composition can be used for the treatment and/or prevention of diseases in mammals, especially humans, possibly including administration of a pharmaceutically effective amount of the pharmaceutical composition to the mammal Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The term “therapeutic effective amount” means an amount which provides a therapeutic benefit for the patient.

The composition of genetically modified T cells obtained by the method of the present invention may be administered either alone, or as a pharmaceutical composition in combination with diluents and/or with other components such as cytokines or cell populations. Briefly, pharmaceutical compositions of the present invention may comprise the genetically modified T cells of the present disclosure, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The term “activation” as used herein refers to inducing physiological changes with a cell that increase target cell function, proliferation and/or differentiation.

The term “transduction” means the transfer of genetic material from a viral agent such as a lentiviral vector particle into a eukaryotic cell such as a T cell.

The tumor associated antigen (TAA) as used herein refers to an antigenic substance produced in tumor cells. Tumor associated antigens are useful tumor or cancer markers in identifying tumor/cancer cells with diagnostic tests and are potential candidates for use in cancer therapy. Preferentially, the TAA may be expressed on the cell surface of the tumor/cancer cell.

The term “removal of modulatory agents” as used herein refers to the physical removal of the modulatory agents from the T cells and/or to the inactivation of the modulatory agent to that effect that it has no effect anymore on the activity of T cells.

Lentivirus is a genus of Retroviridae that cause chronic and deadly diseases characterized by long incubation periods, in the human and other mammalian species. The best-known lentivirus is the Human Immunodeficiency Virus HIV which can efficiently infect nondividing cells, so lentiviral derived retroviral vectors are one of the most efficient methods of gene delivery.

To generate retroviral vectors such as lentiviral vectors the gag/pol and env proteins needed to assemble the vector particle are provided in trans by means of a packaging cell line, for example, HEK 293T. This is usually accomplished by transfection of the packaging cell line with one or more plasmids containing the gag/pol and env genes.

The term “removal of residual lentiviral vector particle” as used herein refers to the physical removal of the residual lentiviral vector particles from the T cells and/or to the inactivation of the residual lentiviral vector particles to that effect that they do not genetically modify T cells anymore

The term “residual lentiviral vector particles” as used herein refer to the portion of lentiviral vector particles that have not transduced T cells in the sample comprising T cells.

The term “the method is performed in equal or less than 144 hours, less than 120 hours, less than 96 hours, less than 72 hours, less than 48 hours, or less than 24 hours” means that the duration of the process as disclosed herein does not take longer than the respective timeframe from the beginning of the process, i.e. the provision of a sample that comprises T cells, to the sample that comprises the genetically modified T cells that subsequent may be ready to (re)-infusion to a patient in need thereof.

In blood, the serum is the component that is neither a blood cell (serum does not contain white blood cells—leukocytes, or red blood cells—erythrocytes), nor a clotting factor; it is the blood plasma not including the fibrinogens. Serum includes all proteins not used in blood clotting and all the electrolytes, antibodies, antigens, hormones, and any exogenous substances. Human serum is the serum from a human

As used herein, the term “subject” refer to an animal. Preferentially, the subject is a mammal such as mouse, rat, cow, pig, goat, chicken dog, monkey or human More preferentially, the individual is a human. The subject may be a subject suffering from a disease such as cancer (a patient), but the subject may be also a healthy subject.

The term “closed system” as used herein refers to any closed system which reduces the risk of cell culture contamination while performing culturing processes such as the introduction of new material, e.g. by transduction, and performing cell culturing steps such as proliferation, differentiation, activation, and/or separation of cells. Such a system allows to operate under GMP or GMP-like conditions (“sterile”) resulting in cell compositions which are clinically applicable. Herein exemplarily the CliniMACS Prodigy® (Miltenyi Biotec GmbH, Germany) is used as a closed system. This system is disclosed in WO2009/072003. But it is not intended to restrict the use of the method of the present invention to the CliniMACS Prodigy®.

The process of the invention may be performed in a closed system (a closed cell sample processing system), comprising a centrifugation chamber comprising a base plate and cover plate connected by a cylinder, pumps, valves, a magnetic cell separation column and a tubing set. The blood samples or other sources comprising T cells may be transferred to and from the tubing set by sterile docking or sterile welding. A suitable system is disclosed in WO2009/072003.

The closed system may comprise a plurality of tubing sets (TS) where cells are transferred between TS by sterile docking or sterile welding.

Different modules of the process may be performed in different functionally closed TS with transfer of the product (cells) of one module generated in the one tubing set to another tubing set by sterile means. For example, T cells can be magnetically enriched in a first tubing set (TS) TS100 by Miltenyi Biotec GmbH and the positive fraction containing enriched T cells is welded off the TS100 and welded onto a second tubing set TS730 by Miltenyi Biotec GmbH for further activation, modification, cultivation and washing.

The terms “automated method” or “automated process” as used herein refer to any process being automated through the use of devices and/or computers and computer software. Methods (processes) that have been automated require less human intervention and less human time. In some instances the method of the present invention is automated if at least one step of the present method is performed without any human support or intervention. Preferentially the method of the present invention is automated if all steps of the method as disclosed herein are performed without human support or intervention other than connecting fresh reagents to the system. Preferentially the automated process is implemented on a closed system such as CliniMACS Prodigy® as disclosed herein.

The closed system may comprise a) a sample processing unit comprising an input port and an output port coupled to a rotating container (or centrifugation chamber) having at least one sample chamber, wherein the sample processing unit is configured to provide a first processing step to a sample or to rotate the container so as to apply a centrifugal force to a sample deposited in the chamber and separate at least a first component and a second component of the deposited sample; and b) a sample separation unit coupled to the output port of the sample processing unit, the sample separation unit comprising a separation column holder, a pump, and a plurality of valves configured to at least partially control fluid flow through a fluid circuitry and a separation column positioned in the holder, wherein the separation column is configured to separate labeled and unlabeled components of sample flown through the column.

Said rotating container may also be used as a temperature controlled cell incubation and cultivation chamber (CentriCult Unit=CCU). This chamber may be flooded with defined gas mixes, provided by an attached gas mix unit (e.g. use of pressurized air/N2/CO2 or N2/CO2/O2).

All agents may be connected to the closed system before process initiation. This comprises all buffers, solutions, cultivation media and supplements, MicroBeads, used for washing, transferring, suspending, cultivating, harvesting cells or immunomagnetic cell sorting within the closed system. Alternatively, such agents might by welded or connected by sterile means at any time during the process.

The cell sample comprising T cells may be provided in transfer bags or other suited containers which can be connected to the closed system by sterile means.

The term “providing a (cell) sample comprising T cells” means the provision of a cell sample, preferentially of a human cell sample of hematologic origin. Normally, the cell sample may be composed of hematologic cells from a donor or a patient. Such blood product can be in the form of whole blood, buffy coat, leukapheresis, PBMCs or any clinical sampling of blood product. It may be from fresh or frozen origin.

The term “washing” means for example the replacement of the medium or buffer in which the cells are kept. The replacement of the supernatant can be in part (example 50% of the medium is removed and 50% fresh medium is added) this often is applied for dilution or feeding purposes, or entirely. Several washing steps may be combined in order to obtain a more profound replacement of the original medium in which the cells are kept. A washing step often may involve pelleting the cells by centrifugation forces and removing the supernatant. In the method of the present invention, cells may be pelleted by rotation of the chamber at e.g. 300×g and the supernatant may be removed during rotation of the chamber. Medium may be added during rotation or at steady state.

Generally, the washing or washing step may be performed once or by a series of media/buffer exchanges (at least twice exchanges, e.g. 2, 3, 4, 5, 6, 7, 8, 9 or 10 exchanges) thereby removing the substances intended to be removed from the T cells such as human serum and/or its components, the magnetic particles or the residual lentiviral vector particles. The exchanges may be performed by separation of cells and media/buffer by centrifugation, sedimentation, adherence or filtration and subsequent exchange of media/buffer.

In general, a CAR may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the invention the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell.

Such a CAR may be also named “anti-tag” CAR or “adapterCAR” or “univerdal CAR” as disclosed e.g. in U.S. Pat. No. 9,233,125B2.

The haptens or tags may be coupled directly or indirectly to a polypeptide (the tagged polypeptide), wherein the polypeptide may bind to said disease associated antigen expressed on the (cell) surface of a target.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA). The CARs of the invention may comprise one or more antigen binding domains (e.g. a tandem CAR). Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art. “Spacer” or “hinge” as used herein refers to the hydrophilic region which is between the antigen binding domain and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (or the intracellular signaling domain) of the CAR is responsible for activation of at least one of the normal effector functions of the immune cell in which the CAR is expressed. “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD).

In some embodiments, the endodomain may contain a primary cytoplasmic signaling domains or a co-stimulatory region, but not both. In these embodiments, an immune effector cell containing the disclosed CAR is only activated if another CAR containing the missing domain also binds its respective antigen.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adaptor molecules could be designed for tumor specificity and would provide options for altering specificity post-adoptive transfer, key for situations of selection pressure and antigen escape. The CARs that may be expressed in the genetically modified T cells obtained by the method as disclosed herein may be designed to comprise any portion or part of the above-mentioned domains as described herein in any order and/or combination resulting in a functional CAR, i.e. a CAR that mediated an immune effector response of the immune effector cell that expresses the CAR as disclosed herein.

### EXAMPLES

### Example 1: Manual Generation of Genetically Engineered T Cells in a Short Period of Time

A sample containing T cells was provided from buffy coat and PBMC were isolated. The blood products were diluted in CliniMACS® buffer in a ratio of 1:2 or 1:3 and 30 mL were layered onto a 15 mL cushion of Pancoll human. The tubes were centrifuged for 30 min at room temperature and 450×g with moderate brakes. After centrifugation, the cells at the interface were carefully sucked off, and washed three times with 50 mL CliniMACS® buffer in order to remove platelets and residual Pancoll. T cells were isolated using CD4 and CD8 specific MicroBeads (Miltenyi Biotec) according to the manufacturer's instructions. T cells were seeded into 24-well plates with 2 mL T cell suspension per well at a concentration of 1×10⁶ cells/mL in TexMACS medium containing human AB serum (10% (v/v) GemCell), IL-7 (10 ng/mL) and IL-15 (5 ng/mL) To activate the T cells T Cell TransAct™ is added to a final dilution of 1:100. After 24h of cultivation in an incubator at 37° C., 5-10% CO2, lentiviral vectors encoding therapeutic CARs are added at a MOI of 2. 24h post transduction an enzyme dextranase was added 1:100 for 1h at 37° C. that specifically degrades the biodegradable linker present in T Cell TransAct™ and Microbeads and both reagents are released from the T cells. Non-cellular components such as remaining lentiviral vectors, degraded components of the T cell TransAct™ and Microbeads are separated from the transduced T cells by centrifugation at 450×g for 10 min. The supernatant is removed and fresh media is added to the same volume. The washing procedure is repeated 3 times to decrease the impurities. The transduced T cells are analysed by flow cytometry to determine the transduction efficiency and perform functional assays such as killing assays in coculture with tumor target cells expressing the CAR antigen.

### Example 2: Automated Generation of Genetically Modified T Cells within a Short Period of Time

A sample of T cells is provided in bag derived from a leukapheresis from a donor. The bag is connected by sterile welding to a tubing set installed on the CliniMACS Prodigy® device. CliniMACS buffer, CliniMACS CD4 and CD8 reagents (Miltenyi Biotec GmbH) as well as activating reagent are also connected to the same Tubing set. Within the fully automated process, the enrichment step is launched that takes in total 30 min to 2h. In detail, the tubing set is automatically primed with buffer, then the leukapheresis product is transferred to the chamber of the tubing set where it is washed 3 times with CliniMACS buffer in order to remove serum and platelets. The cells are magnetically labelled with CliniMACS CD4 and CD8 reagents and trapped onto a column placed in a magnetic field. The labeled cells trapped onto the column are rinsed several times and eluted into the target cell fraction bag. Part of the enriched cells are transferred to the CentriCult™ Chamber via sterile welding connection and formulated in MACS GMP TexMACS medium supplemented with IL-7/IL-15 (all Miltenyi Biotec GmbH). Within the automated process the activation step is started and the activation reagent MACS GMP TransAct is automatically added to the culture. After enrichment and up to 24 hours, a bag containing lentiviral vector is sterile welded onto the tubing set and the lentiviral vector suspension is transferred into the CentriCult™ Chamber containing the activated T cells. 24 hours to 48 hours the activation reagent and magnetic particles are degraded by adding an dextranase, specific for the biodegradable linker present in the activation reagent and magnetic particles. After 1 h, non-cellular components such as residual lentiviral vectors, degrading enzymes and degraded components of the activation reagent and CliniMACS CD4 and CD8 reagents are removed by washing. The genetically modified T cells are automatically formulated in a solution suitable for human infusion.

### Example 3: Administration of CAR T Cells with Additional Cleanup Steps

CAR T cell therapy is provided e.g. to treat pediatric and adult patients with relapsed or refractory CD19 positive B cell malignancies. The clinical method of preparing the genetically engineered T cells is based on example 2, whereby patient cells (derived from BM, blood or leukapheresis) are connected to the CliniMACS Prodigy® device and processed rapidly (i.e. preferably less than 24h) and reinfused into the patient. The duration of the process can be modulated to match timing for required patient preparative regimen (e.g. chemotherapeutic treatment to lymphodeplete), meet medical needs and clinical applicability (e.g. clinical protocol, patient health status, reactivity of the doctors, hospital stay). Advantages of the described invention are to enable rapid treatment and patient care as well as to enable “bed side” preparation of drug products. The invention describes a solution for such rapid cell preparation where by potentially harmful substances such as viral vectors and activation reagents are removed prior to infusion. For example, remaining activation reagents contaminating the drug product may be harmful upon infusion as they may lead to activation of T cells in vivo. This may lead to a rapid release of proinflammatory cytokines, causing severe cytokine release syndrome, fever, hypotension, organ failure and even deaths.

In addition, remaining lentiviral vectors contaminating the drug product in soluble and/or cell bound form may be harmful upon infusion as they may provoke an unwanted immune response such as complement activation, antibody-dependent cell-mediated cytotoxicity, inducing an adoptive immune response against antigens delivered by the lentiviral vector and/or transduction of non-target cells in vivo. The transduction of non-target cells and the subsequent expression of the transgene may induce unwanted side-effects such as the induction of unwanted immune responses, oncogenicity, altered survival, proliferation, physiological state and natural function.

### Example 4: Setting Up the Process for the Genetic Engineering of T Cells in 3 Days

T cells from 2 healthy donors were enriched untouched with the Pan T cell isolation kit, human (Miltenyi Biotec) and polyclonally stimulated on day 0 with T Cell TransAct™ (Miltenyi Biotec)—a modulatory reagent comprising an antibody or antigen binding fragment thereof specific for CD3 and an antibody or antigen binding fragment thereof specific for CD28 coupled directly or indirectly to a biodegradable linker. The stimulated T cells were transduced on the same day or day 1 with VSV-G pseudotyped GFP encoding LV at a MOI of 2 in 24 wells at 1e6 cells/ml. in IL-7/IL-15 containing TexMACS media. On day 1, 2 or 3, dextranase specifically digesting the biodegradable linker was added and the transduction efficiency was evaluated on day 10 by flow cytometry. T cells stimulated on day 0 and transduced on day 1 without adding dextranase served as control for the conventional protocol for the genetic engineering of T cells. As depicted in FIG. 9, T cells transduced on day 0 and incubated with the enzyme specific for the biodegradable linker showed the lowest transduction efficiency levels indicating insufficient T cell stimulation. This was confirmed by analyzing T cells that were stimulated longer by adding later on day 2 or 3 and higher transduction efficiency levels were detectable as compared to T cells incubated with the enzyme on day 0. Higher transduction efficiencies that were close to the conventional protocols were observed for stimulated T cell that were transduced on day 1 and incubated with dextranase on day 2 or 3.

### Example 5: Removal of the Modulatory Agent in the CliniMACS® Prodigy System for the Genetic T Cell Engineering within 3 Days and Analysis of the T Cell Activation Levels

A leukapheresis sample of a healthy donor with up to 1e9 CD4/CD8 cells was automatically processed in the CliniMACS® Prodigy system to generate CAR T cells within 3 days. On day 0, a bag containing the leukapheresis sample was sterile connected to the CliniMACS Prodigy® Tubing Set 520 by welding. The cells were automatically washed and labelled with CD4 and CD8 CliniMACS reagent to enrich T cells. 4e8 T cells were transferred in IL-7/IL-15 containing medium to the centrifugation and cultivation chamber and were polyclonally stimulated with the modulatory agent MACS® GMP T Cell TransAct™ (Miltenyi Biotec) in a cultivation volume of 200 ml. On day 1, the isolated and activated T cells were genetically modified with VSV-G pseudotyped lentiviral vectors with a MOI of 3 to induce the expression of CD20/CD19 specific tandem CAR. A bag containing 10 ml of lentiviral vectors was sterile connected to the tubing set and automatically transferred to the chamber containing the T cells. On day 2, 10 ml of a solution containing the enzyme specific for the biodegradable linker was sterile connected to the tubing set and automatically added to the chamber containing the T cells to specifically degrade the linker, thereby the antibodies or fragments specific for CD3 and CD28 are released and the activity of the modulatory agent is inhibited. As control, a CliniMACS® Prodigy system run was performed under the same conditions and the same donor material but without the addition of the enzyme specific for the biodegradable linker. After washing multiple times a cell product was obtained that is suitable for therapeutic application. The presence of the biodegradable linker was assessed for both T cell engineering runs in the CliniMACS® Prodigy system by flow cytometry on the formulated cells by staining with antibodies specific for the biodegradable linker. As depicted in FIGS. 10A and 10B, the biodegradable linker was efficiently removed in the CliniMACS® Prodigy system as only a minor fraction of linker positive cells was detectable when compared to the CliniMACS® Prodigy run without added enzyme. In addition, the mean intensity levels (MFI) for the biodegradable linker for all viable cells was at background levels when the enzyme was added (see FIG. 10B). In contrast, high mean intensity levels (MFI) were present for the CliniMACS® Prodigy run without added enzyme.

The impact of removing the modulatory agent on the stimulation was evaluated by flow cytometry upon staining for CD25 and CD69 as both are described to be reliable T cell activation markers (CD25: clone REA570 and CD69: REA824 (both Miltenyi Biotec): CD69 an earlier activation marker than CD25. Non-stimulated T cells obtained from the same donor from small scale cultures served as control and harvested T cells from the CliniMACS® Prodigy system treated with or without enzyme were analyzed. Compared to the non-stimulated control cells, highly elevated mean intensity levels for both activation markers were detected for both T cell engineering conditions (see FIG. 11). This confirmed that the stimulation until day 2 was already sufficient to induce upregulation of both activation markers. This also indicates that the modulatory agent may be removed already at day 2 without affecting the stimulation.

### Example 6: Assessing the Expansion Potential of Stimulated T Cells in the CliniMACS® Prodigy System for the Genetic Engineering of T Cells within 3 Days

Multiple manufacturing runs with stimulated T cells were performed as described in Example 5 in the presence of dextranase added on day 2 but with varying starting T cell numbers ranging from 1e8 to 4e8. The input T cell number on day 0 was compared to the output T cell number obtained on day 3. T cell expansion was not detectable on day 3 suggesting that the T cells were sufficiently stimulated but proliferation has not started yet (see also FIG. 12). In consequence, the manufacturing protocol for the genetic modification of T cells within 3 days is too short to support T cell proliferation in vitro. The data also suggests that the yield of harvestable CAR T cells is efficiently increased by increasing the starting cell number.

### Example 7: Evaluating the CAR Expression Kinetics in Small Scale and in the CliniMACS® Prodigy System

CAR expression kinetics are especially crucial for the success of CAR T cell therapy when short manufacturing processes are applied. Infused CAR T cells that express the therapeutic CAR molecule not sufficiently remain non-functional because the tumor antigen cannot be recognized. During this time the tumor progression may continue within the patient making it more challenging for the CAR T cells to scope with the higher tumor burden. To date, the most prevalent adverse effect following infusion of CAR T cells is the onset of immune activation, known as cytokine release syndrome (CRS). It is a systemic inflammatory response caused by cytokines released by infused CAR T cells shortly after infusion recognizing a potentially high load of tumor cells expressing the CAR antigen. CAR T cell manufacturing within a short period of time may at least partially reduce this toxicity because not all CAR T cell express the CAR at this early time point and at high CAR expression levels.

For small scale studies, CD4/CD8 enriched T cells from 2 healthy donors were polyclonally stimulated with T Cell TransAct™ (Miltenyi Biotec). On day 1 the stimulated T cells were transduced with CAR encoding LV at a MOI of 9 in 24 wells and 1e6 cells/ml and the kinetic of CAR expression was determined by flow cytometry until day 13 as ratio of transduced cells (i.e. transduction efficiency) with CAR detection reagents comprising the CAR antigen peptide directly or indirectly coupled to PE (e.g. CD19 CAR Antibody, anti-human, 130-115-965, Miltenyi Biotec). As depicted in FIG. 13, 2 days post transduction 16% of the T cells were CAR positive but a distinct population expressing the CAR was not detectable yet. Upon day 5 the transduction efficiency levels reached plateau levels at 18-22% with a distinct CAR expressing population.

For the studies in large scale in the CliniMACS® Prodigy system 2e8 CD4, CD8 enriched T cells were polyclonally stimulatd on day 0 with MACS® GMP T Cell TransAct™ (Miltenyi Biotec) in 100 ml of IL-7/IL-15 containing medium in the cultivation chamber. On day 1, the isolated and stimulated T cells were genetically modified with VSV-G pseudotyped CD19 CAR encoding lentiviral vectors with a MOI of 62.5 by sterile connecting a LV containing bag to the tubing set.

On day 2, 10 ml of a solution containing dextranase specific for the biodegradable linker of the modulatory agent was sterile connected and automatically added to the chamber containing the T cells. On day 3 a sample of the cell suspension was analyzed by flow cytometry after staining with CAR detection reagents comprising the CAR antigen peptide directly or indirectly coupled to PE (e.g. CD19 CAR Antibody, anti-human, 130-115-965, Miltenyi Biotec). As depicted in FIG. 14, 2 days post transduction 19% of the T cells were CAR positive. The cultivation process within CliniMACS® Prodigy was prolonged to enable analysis at later time points. The transduction efficiency increased to 75% on day 10 indicating that the CAR is not yet sufficiently expressed 2 days after transduction.

### Example 8: Optimizing CAR T Cell Manufacturing Parameters in the CliniMACS® Prodigy System

The manufacturing process of CAR T cells is a complex process dependent on multiple parameters and with a high degree of donor variation. Optimizing the gene transfer efficiency and T cell cultivation offers the possibility to reduce the amount of lentiviral vector needed and to obtain a higher number of (CAR) T cells. In two separate T cell engineering runs 1e8 or 4e8 CD4, CD8 enriched T cells were polyclonally stimulated on day 0 with MACS® GMP T Cell TransAct™ (Miltenyi Biotec) in IL-2 containing medium in the CliniMACS® Prodigy system. On day 1, the isolated and stimulated T cells were genetically modified with 2.5 ml of VSV-G pseudotyped CD20/CD19 tandem CAR encoding lentiviral vectors for 1e8 T cells (see FIG. 15: Condition I) and in parallel with the same volume for 4e8 T cells. For the 4E8 CAR T cell manufacturing run the process activity matrix was additionally modified to enable cultivation at higher cell densities by increasing the volume and by implementing early shaking steps directly after adding the lentiviral vector volume (see FIG. 15: Condition II). On day 2, the same volume of dextranase was applied to both T cell manufacturing runs by sterile connection and automatic addition to the cultivation chamber. On day 3, the manufactured T cells were washed multiple times, harvested and the total T cell number was determined by cell counting. A washed and harvested cellular sample of both CAR T cell manufacturing runs was cultivated for another 8 days in 24 wells in the incubator to enable reliable assessment of the transduction efficiency at later time points with CAR detection reagents comprising the CAR antigen peptide directly or indirectly coupled to PE (e.g. CD19 CAR Antibody, anti-human, 130-115-965, Miltenyi Biotec). As depicted in FIG. 15A, the transduction efficiency was 32% for condition II, whereas the transduction efficiency for condition I was only 20%—albeit a higher LV dose per cell (MOI) was applied for condition I. This indicates that the parameters of condition II favor higher frequencies of CAR T cells underlining the potential of optimization the CAR T cell manufacturing protocol. For condition II not only a higher transduction efficiency was determined but also 4e8 T cells were transduced. This increased the total number of CAR transduced T cells almost 7 fold for condition II when compared to condition I.

### Example 9: The In Vitro Function of CAR T Cells Generated within 3 Days

The function of CAR T cells is typically evaluated in vitro upon coculturing with tumor cells expressing the CAR antigen. Within a short period of time and upon CAR antigen contact, CAR T cells release inflammatory cytokines such as Interferon-gamma (IFN-g), Granulocyte-macrophage colony-stimulating factor (GM-CSF) and IL-2. In addition granzyme B and perforin B is released and the number of viable tumor cells is reduced. These functional assays were performed to characterize the functionality of CAR T cells manufactured within 3 days. T cells from 2 healthy donors were enriched untouched with the Pan T cell isolation kit, human (Miltenyi Biotec) and polyclonally stimulated on day 0 with T Cell TransAct™ (Miltenyi Biotec)—a modulatory agent comprising an antibody or antigen binding fragment thereof specific for CD3 and an antibody or antigen binding fragment thereof specific for CD28 coupled directly or indirectly to a biodegradable linker. The stimulated T cells were transduced on day 1 with VSV-G pseudotyped CD20 CAR encoding LV at a MOI of 10 in 24 wells at 1e6 cells/ml in IL-7/IL-15 containing TexMACS media. On day 2 dextranase was added and the transduced T cells were washed and harvested on day 3 to setup the coculture at different effector to target ratios (E:T). The transduction efficiency was 70% at day 3 as measured by flow cytometry using a CAR specific detection reagent. 50000, 17000, 6000 or 2000 total T cells were added to 40000 CD20 expressing Raji cells in triplicate in 96 Well flat bottom plates in RPMI/10% FCS/L-Glutamin media. Transgenic Raji cells expressing GFP (Raji-GFP) were used to enable identification and quantification of the tumor cells in the coculture by flow cytometry to determine the cytotoxic activity of the manufactured CAR T cells. As control, cocultures with Raji-GFP cells were established in triplicate in parallel with non-stimulated, non-transduced T cells. In addition, tumor cells were cocultivated with stimulated but non-transduced T cells. This way potentially unspecific, cytotoxic activity is easily detected. 24h post coculture setup, 100 μl of supernatant were taken from each well to evaluate the cytokine expression levels by flow cytometry using the MACSPlex Cytokine Kit Assay (Miltenyi Biotec). For CD20 CAR transduced T cells generated within 3 days, IFN-g, GM-CSF and IL-2 levels were detectable at high levels even beyond the level of quantification in an E:T dependent manner (see FIG. 16). In contrast, no cytokines were detectable for non-stimulated T cells and for stimulated T cells that remained untransduced. This confirms the specific antitumoral response of CAR transduced T cells that were manufactured within 3 days.

Cocultured T and Raji-GFP cells were cultivated for another 2 days when 50% of the cells were analyzed by flow cytometry to quantify the number of remaining tumor cells and consequently the CAR T cell potency (round 1; left). Another 20,000 Raji-GFP tumor cells were added to the remaining 50% of the coculture to evaluate the potency of the CAR T cells when more tumor cells are present resembling conditions to be challenging for CAR T cells. After another 72h flow cytometry was performed to quantify the number of remaining tumor cells of the second round of coculture (round 2: right). For a high E:T ratio of 1.25:1 almost 100% of the Raji cells were killed in the first and the second round of coculture (see FIG. 17). In contrast only 50% and 40% of the target cells were detectable for the untransduced control in the first and second coculture. For a E:T ratio of 0.425:1 a comparable functionality pattern was detectable as for 1.25:1 but at lower overall levels. 60% of the tumor cells were lysed in the presence of CAR transduced T cells in the first and second round of coculture. In contrast for the untransduced controls 40% of the tumor cells were lysed in the first round and no killing was measured in the second round. For E:T ratios of 0.15:1 the frequency of T cells was too low to induce the cytotoxic activity against the Raji-GFP tumor cells. In summary, the functionality of CAR T cells generated within 3 days was confirmed by in vitro assays showing cytokine release and specific killing for CAR transduced T cells.

### Example 10: In Vivo Function of CAR T Cells Generated within 3 Days

The in vivo functionality of CAR transduced T cells generated within 3 days was confirmed in 6 to 8 week old NOD scid gamma (NSG) (NOD.Cg-Prkdc^{scid}I12rg^{tm1Wjl}/SzJ) mice. All experiments were performed in compliance with the “Directive 2010/63/EU of the European Parliament and of the Council of Sep. 22, 2010 on the protection of animals used for scientific purposes” and in compliance with the regulations of the German animal protection law.

Briefly, a leukapheresis sample of a healthy donor was automatically processed in the CliniMACS® Prodigy system to generate CAR T cells within 3 days (see FIG. 18 top). On day 0, a bag containing the leukapheresis sample was sterile connected to the CliniMACS Prodigy® Tubing Set 520 by welding. The cells were automatically washed and labelled with CD4 and CD8 CliniMACS reagent to enrich T cells. 2e8 T cells were transferred in IL-7/IL-15 containing medium to the cultivation chamber and were polyclonally stimulated with MACS® GMP T Cell TransAct™ (Miltenyi Biotec) in a cultivation volume of 200 ml. On day 1, the isolated and activated T cells were genetically modified with VSV-G pseudotyped lentiviral vectors to induce the expression of CD22/CD19 Tandem-CAR. A bag containing 10 ml of lentiviral vectors was sterile connected to the tubing set and automatically transferred to the chamber containing the T cells. On day 2, 10 ml of a solution containing dextranase were sterile connected to the tubing set and automatically added to the chamber containing the T cells to specifically degrade the linker, thereby the antibodies or fragments specific for CD3 and CD28 are released and the activity of the modulatory agent is inhibited. After washing multiple times the cell product was analyzed by flow cytometry to determine the transduction efficiency, viability and cellular composition at each step (see FIG. 19). The cellular composition was determined upon staining for CD45h, CD3, CD4, CD8, CD16/CD56, 7-AAD, CD19, CD14. After formulation 67% CD4 T cells, 18% CD8 T cells and 7% NKT cells were detected. The frequency of NK cells, eosinophils, neutrophils, B cells or monocytes was at minimum level of detection. The transduction efficiency was determined by flow cytometry with CAR detection reagents comprising the CAR antigen peptide directly or indirectly coupled to PE (e.g. CD19 CAR Antibody, anti-human, 130-115-965, Miltenyi Biotec). At the day of harvest the transduction efficiency was 21%. This increased to 73% when analyzed after an extended cultivation in small scale for another 8 days when stable CAR expression levels were reached. Raji tumors have been established by intravenous inoculation with 5e5 Firefly luciferase-expressing Raji cells 4d days before harvesting the genetically engineered T cells (see FIG. 17). 3e6 or 6e6 total T cells from the CAR transduced groups were injected per mouse at the harvesting day (see FIG. 18 bottom) with 7 mice per group. Two additional groups were established as negative control: one group received 5e5 tumor cells but no T cells (n=7; tumor only) and one group received 5e5 tumor cells and 3e6 not transduced T cells (n=7) from the same donor cultivated in parallel in small scale. Tumor growth as well as anti-tumor response was monitored frequently using an In vivo Imaging System (IVIS Lumina III). For this purpose, 100 μl XenoLight Rediject D-Luciferin Ultra was injected i.p. and subsequently mice were anesthetized using the Isofluran XGI-8 Anesthesia System. Measurement was performed six min after substrate injection.

All mice are shown for the group that received 3e6 untransduced and 3e6 transduced T cells (see FIG. 20). 3 representative mice out of 7 mice are shown for the group that received no T cells (i.e. tumor only). Tumor burden increased rapidly for all mice that received untransduced T cells or no T cells. The increase in tumor burden over time is comparable for both control groups. Mice in both control groups had to be sacrificed 14d post T cell injection before reaching critical tumor burden levels. In contrast, mice in groups that have received CAR transduced T cells manufactured within 3 days showed a decelerated increase at early time points in an dose-dependent manner 3 and 7 days post T cell injection when compared to the control groups. The level of tumor burden for the CAR transduced T cell groups peaked on day 7 post T cell injection. The tumor progression was completely reversed as detected by a steady and uniform reduction of the tumor burden for all mice to levels that were initially measured at the start of the experiment. Representative in vivo imaging data is shown for all mice in the groups that received 3e6 CAR transduced T cells and the same dose of untransduced T cells. Representative mice are shown for the tumor only group.

The tumor burden is depicted as mean and SEM for all groups in FIG. 21 including also the group that has received the highest dose with 6E6 CAR transduced T cells per mouse group (n=7). As expected the 6e6 group the quickest antitumoral response but the tumor burden at the end of the experiment was comparable to 3e6 CAR T cell group. This data confirms that CAR T cells generated within 3 days and without any expansion are mediating potent antitumoral responses. This result was confirmed by flow cytometry data to quantify human tumor cells and human T cell subsets in spleen, bone marrow and blood. 3 randomly selected mice from the “Tumor only” and “3E6 untransduced T cells” control groups were sacrificed on day 14 and the abundance of human cells, Raji cells and T cells subset in these organs was quantified by staining for CD45h, CD4, CD8, CD20, CD22, 7-AAD, CD19 CAR Detection (all Miltenyi Biotec). The mice in the CAR transduced T cells groups were analyzed analogously when 3 out 7 mice were randomly selected and sacrificed on day 18. As expected no T cells were found in the Tumor only group (see FIG. 22). Only up to 20% T cells were detectable for non-transduced cohort. In contrast, the frequency of human T cells was highest with up to 75% in the cohort containing mice that were infused with CAR transduced T cells. Thus T cells are more abundant in the cohort containing the transduced T cells than in the cohort with the untransduced T cells. This indicates the CAR T cells were capable of expanding and persist. This is in line data shown in FIG. 21, showing that CAR transduced T cells are capable of controlling the tumor, whereas the abundance of non-transduced T cells was relatively low and not able to control tumor outgrowth.

FIG. 23 further demonstrated the effect of non-transduced on Tumor still being present in contrast to transduced group where tumor cells are gone and shows also that CD8 expanded more

The cellular composition of the human subset was investigated in more detail by determining the frequency of cell subsets for human cells only (see FIG. 23). Again, no human T cells were found in the tumor only groups. 20-60% of the human cells were remaining Raji cells for the untransduced T cell group with a CD4 to CD8 ratio of 2:1 to 3:1. For the CAR transduced T cells groups about 50% human CD4 and 50% human CD8 T cells were found and values close to background level were detectable for the remaining Raji cells in the CAR transduced group. This data suggest specific expansion of the CD8 T cell subset in vivo for to the cohort containing mice with CAR transduced T cells.

When analyzing the spleen, no human T cells were found in this lymphocytic organ for the tumor only cohort (see FIG. 24). T cell frequencies up to 10% were determined for the cohort containing untransduced T cells. In contrast the frequency of human T cells was much higher for the CAR transduced T cell group with up to 40% human T cells confirming T cell expansion and antitumoral activity as measured by the in vivo imaging data.

In summary the in vivo data confirms the in vitro functionality data and shows that the untransduced T cells were not able to control the outgrowth of Raji cells. In contrast, the highest fraction of human T cells was found in bone marrow (which is the preferred niche of the Raji engraftment and expansion), showing that the 3 day expanded CAR T cells are also capable to home to such niches and promote antitumoral activity.

Thus, CAR T cells generated within 3 days even in the absence of an explicit expansion step surprisingly promote robust antitumoral activity in vitro and in vivo proving that in vivo expansion but not in vitro expansion is essential for the generation of functional CAR T cells.

1. A method for the generation of genetically modified T cells comprising the steps
a) a sample provided, said sample comprising T cells
b) preparation of said sample by centrifugation
c) enrichment of the T cells of step b
d) activation of the enriched T cells using modulatory agents
e) genetic modification of the activated T cells by transduction with lentiviral vector particles
f) removal of said modulatory agents,
thereby generating a sample of genetically modified T cells,
wherein said method is performed in equal or less than 144 hours, less than 120 hours, less than 96 hours, less than 72 hours, less than 48 hours, or less than 24 hours.

2. The method according to claim 1, wherein said sample of step a) comprises human serum and wherein said serum is removed in step b).

3. The method according to claim 1, wherein the T cells are enriched in step c) for CD4 and/or CD8 positive T cells by using CD4 and/or CD8 as positive selection marker, and/or depleted of cancer cells by using a tumor associated antigen (TAA) as a negative selection marker.

4. The method according to claim 3, wherein said enrichment of CD4 and/or CD8 positive T cells is performed by magnetic cell separation steps comprising:
i) contacting the T cells with magnetic particles that are directly or indirectly coupled to antibodies or antigen binding fragments thereof specific for CD4 and/or CD8, wherein said magnetic particles and said antibodies or antigen binding fragments thereof coupled thereto can be removed
ii) separating the CD4 and/or CD8 T cells in a magnetic field
iii) removal of said magnetic particles from the enriched T cells after the separation.

5. The method according to claim 4, wherein said enrichment of CD4 and/or CD8 positive T cells is performed by magnetic cell separation steps comprising:
i) contacting the T cells with magnetic particles that are directly or indirectly coupled to antibodies or antigen binding fragments thereof specific for CD4 and/or CD8, wherein said magnetic particles and said antibodies or antigen binding fragments thereof coupled thereto can be disrupted chemically and/or enzymatically
ii) separating the CD4 and/or CD8 T cells in a magnetic field
iii) removal of said magnetic particles from the enriched T cells after the separation step by chemical and/or enzymatical disruption of said magnetic particles and said antibodies or antigen binding fragments thereof coupled thereto.

6. The method according to claim 1, wherein said modulatory agents comprise an antibody or antigen binding fragment thereof specific for CD3 and/or an antibody or antigen binding fragment thereof specific for CD28 coupled directly or indirectly via a linker, wherein said antibodies or antigen binding fragments thereof specific for CD3 and CD28 can be removed.

7. The method according to claim 1, wherein said modulatory agents comprise an antibody or antigen binding fragment thereof specific for CD3 and/or an antibody or antigen binding fragment thereof specific for CD28 coupled directly or indirectly via a linker, wherein said antibodies or antigen binding fragments thereof specific for CD3 and CD28 can be disrupted chemically and/or enzymatically, and wherein said modulatory agents are removed by chemical and/or enzymatical disruption of said antibodies or antigen binding fragments thereof specific for CD3 and CD28.

8. The method according to claim 1, wherein said modulatory agents comprise an antibody or antigen binding fragment thereof specific for CD3 and/or an antibody or antigen binding fragment thereof specific for CD28 that are directly coupled via a biodegradable linker, wherein said biodegradable linker is degraded by adding an enzyme that specifically digests the glycosidic linkages of said biodegradable linker.

9. The method according to claim 8, wherein said biodegradable linker is or comprises a polysaccharide and said enzyme that specifically digests the glycosidic linkages is a Hydrolase.

10. The method according to claim 1, wherein after the genetic modification of the T cells by transduction with lentiviral vector particles residual lentiviral vector particles are removed.

11. The method according to claim 10, wherein said removal of residual lentiviral vector particles is performed by washing, wherein the washing results in an at least 10-fold, preferably 100-fold reduction of residual vector particles in the sample that comprises the genetically modified T cells.

12. The method according to claim 10, wherein said removal of residual lentiviral vector particles is performed by incubation with substances that inactivate lentiviral vector particles and/or reduce their stability.

13. The method according to claim 2, wherein the removed human serum or isolated substances therefrom that inhibit productive transduction of lentiviral vector particles to T cells is added to the genetic modified T cells, thereby removing and/or neutralizing residual lentiviral vector particles.

14. The method according to claim 1, wherein said method is an automated method performed in a closed system.

15. The method according to claim 1, wherein the number of T cells in said generated sample is less than 10-fold higher compared to the number of T cells in said provided sample.
